(19) United States
(12) Patent Application Publication
(10) Pub. No.: US 2021/0015414 A1
(43) Pub. Date: Jan. 21, 2021

(54) SYSTEM AND METHOD FOR BLOOD SAMPLE COLLECTION AND PROCESSING

(71) Applicant: DH Diagnostics LLC, Brea, CA (US)

(72) Inventors: David L. YANG, Orange, CA (US); Fiona S. ADAIR, Brea, CA (US); Dirk HECKEL, Yorba Linda, CA (US)

(21) Appl. No.: 16/932,385

(22) Filed: Jul. 17, 2020

Related U.S. Application Data
(60) Provisional application No. 62/876,078, filed on Jul. 19, 2019.
(60) Provisional application No. 62/876,069, filed on Jul. 19, 2019.
(60) Provisional application No. 62/876,058, filed on Jul. 19, 2019.

(51) Int. Cl. A61B 5/15; G01N 33/52; G01N 33/49

(52) U.S. Cl. CPC A61B 5/150755; A61B 5/150786; G01N 33/491; G01N 33/52

(57) ABSTRACT

Blood sample collection and processing system (1) using a tube (102) and a holder (104) is described herein. The tube (102) includes a plasma separation system (114) for converting a whole blood sample (129) into a plasma sample without using centrifugation. The plasma separation system (114) may comprise a filtration system such as a hollow fiber element (115). The tube (102) is thereafter placed in the holder (104) for analysis regarding the plasma. The results of the analysis may be displayed to the user via a display screen (134) to allow the user to determine whether the sample is sufficient for further use. Inasmuch as the analysis system (132) performs a rapid analysis of the plasma sample, the patient may still be present or in close proximity and if the sample is insufficient, the healthcare provider can take another whole blood sample (129), counsel the client regarding the blood sample (129), or examine the patient further.

### CROSS-REFERENCE TO RELATED APPLICATIONS

This patent application claims the benefit of priority to U.S. Provisional Application Ser. No. 62/876,058, filed on Jul. 19, 2019; U.S. Provisional Application Ser. No. 62/876,069, filed on Jul. 19, 2019; and U.S. Provisional Application Ser. No. 62/876,078, filed on Jul. 19, 2019, the disclosures of which are hereby incorporated by reference herein in their entireties for all purposes.

### BACKGROUND

Oftentimes it is desirable to perform a full biochemical analysis on a whole blood sample taken from a patient. However, prior to the initiation of the full biochemical analysis, pre-analytical steps are taken to determine sufficiency of the whole blood sample. For example, the plasma of the whole blood sample may contain a large number of interferents rendering the whole blood sample insufficient for full biochemical analysis. The pre-analytical steps, from blood collection, to centrifugation of the sample, to examination is laborious, time-consuming, and error-prone. Oftentimes, upon a determination that the whole blood sample is insufficient, the patient has left the healthcare facility and must return to the healthcare facility to provide another whole blood sample.

Further, the current process for pre-analytics of blood testing is manual in nature, which is time-consuming and introduces risk of poor sample quality and handling. A phlebotomist draws blood into a single glass or plastic tube through a rubber topped cap via vacuum or low pressure within the tube. The blood sample is transferred to the lab where a clinician processes the sample through multiple steps before the sample is loaded onto a rack of an analyzer instrument. The clinician needs to centrifuge, de-cap, label, scan, order, and rack the sample. The workflow contains about 10-12 steps. Additionally, each step to the process can vary in length with some steps taking up to 1 hour. These lengthy steps are often cut short due to time constraints which may impact the final results. In the occasion that a patient needs multiple tests, various sample types may need to be prepared by repeating this process; not only is this laborious but it can introduce sample handling errors. If there were a more streamlined approach, clinicians would save a lot of time and results would be more reliable.

Current solutions to the reliability problem are directed to improvements on the tube technology, but no current solutions eliminate the need to manually process the sample. Current solutions only make minor improvements to sample quality. Workflow for labs remain cumbersome, time consuming, and more prone to error than proposed solution.

In some cases, a whole blood sample is provided to a lab where a clinician processes the whole blood sample through multiple steps before the sample or derivatives thereof are loaded onto a rack of an analyzer instrument.

Many diagnostic tests are performed on plasma and/or serum samples rather than a whole blood sample. Plasma and/or serum creation is primarily achieved via centrifugation. For most modest throughput labs, this is done manually, whereas high throughput labs use automation lines containing a centrifugation station. This need to centrifuge is time consuming and usually requires batching. Centrifugation on automation tracks is a driver of reduced reliability of the overall automation system.

Therefore, there is a need in the field for simplified workflow and improved reliability regarding generating plasma and/or serum samples from whole blood. In some instances, allowing for random access of the plasma and/or serum samples while keeping a high throughput is desired. Other needs relate to reducing the amount of time required to achieve a result, reducing the price per test, and increasing the reliability of the plasma and/or serum generation.

### SUMMARY

According to a first aspect there is provided a collection system. The collection system includes a tube and a holder. The holder is configured to receive the tube therein.

According to a second aspect there is provided a collection system. The collection system includes a tube having a collection chamber, a plasma separation system, and an analysis pocket extending from the collection chamber. The plasma separation system is disposed in the collection chamber. The analysis pocket is configured to receive therein a unit of plasma provided from the plasma separation system. The collection system further includes a holder configured to draw power from a power supply. The holder includes a display screen, a tube receptacle, an analysis system, a labelling system, and a pressure system. The display screen is powered by the power supply. The tube receptacle is configured to receive the tube therein. The analysis system is configured to collect a set of data from the unit of plasma disposed in the analysis pocket. The analysis system is configured to actuate the display screen to display the set of data. The labeling system is configured to apply a label to the tube. The pressure system is configured to force fluid through a hollow fiber.

The plasma separation system of the collection system according to the second aspect can covert a whole blood sample into a plasma sample without using centrifugation. The plasma separation system may comprise a filtration system such as a hollow fiber element. The tube is thereafter placed in the holder for analysis regarding the plasma. The results of the analysis may be displayed to the user via a display screen to allow the user to determine whether the sample is sufficient for further use. Inasmuch as the analysis system performs a rapid analysis of the plasma sample, the patient may still be present or in close proximity and if the sample is insufficient, the healthcare provider can take another whole blood sample, counsel the client regarding the blood sample, or examine the patient further.

According to a third aspect there is provided a method. The method includes placing a blood sample into a tube. The method further includes creating a plasma sample from the blood sample within the tube. The plasma sample is created without centrifugation. The method further includes receiving the tube in a holder. The method further includes conducting spectroscopic analysis on the sample of plasma with an analysis system of the holder.

According to a fourth aspect there is provided a device. The device includes a main body, a needle attachment feature, a first chamber, a second chamber, and a separation element. The needle attachment feature is configured to couple a needle element to the main body. The needle attachment feature is configured to transmit a blood sample from the needle element into the main body. The separation element is configured to separate the blood sample into the first portion and the second portion. The separation element is configured to transmit the first portion into the first chamber. The separation element is configured to transmit the second portion into the second chamber.

The device according to the fourth aspect can streamline a process of collecting a blood sample and separating the blood sample into different portions. In some cases, the separation element of the device can quickly produce plasma without centrifugation.

According to a fifth aspect there is provided a method. The method includes drawing a blood sample into a device. The method further includes separating the blood sample into a sample of whole blood, a sample of plasma, and a sample of serum. The method further includes transmitting the sample of whole blood into a first chamber connected to the device. The method further includes transmitting the sample of plasma into a second chamber connected to the device. The method further includes transmitting the sample of serum into a third chamber connected to the device.

According to a sixth aspect there is provided a system. The system includes a device having a main body, a needle attachment feature, and a separation element. The needle attachment feature is configured to selectively couple a needle element to the main body. The needle attachment feature is configured to transmit a blood sample from the needle element into the main body. The separation element is disposed inside the main body. The separation element is configured to receive the blood sample and separate the blood sample into a sample of whole blood, a sample of plasma, and a sample of serum.

The system further includes a first sample tube, a second sample tube, and a third sample tube. The first sample tube is removably connected to the device. The first sample tube is configured to receive the sample of whole blood from the device. The second sample tube is removably connected to the device. The second sample tube is configured to receive the sample of plasma from the device. The third sample tube is removably connected to the device. The third sample tube is configured to receive the sample of serum from the device.

According to a seventh aspect there is provided an analyzer. The analyzer is configured to receive a tube containing a blood sample. The analyzer includes a separation system, a transfer element, and a movement system. The transfer element is configured to obtain at least a portion of the blood sample from the tube. The transfer element is configured to deposit the portion of the blood sample into the separation system. The movement system is configured to move the transfer element between the tube and the separation system. The separation system is configured to separate the portion of the blood sample into a separated sample. The sample comprises one of a plasma sample and a serum sample.

The separation system of the analyzer according to the seventh aspect can create a plasma sample or a serum sample from a whole blood sample without centrifugation. The separation system may comprise a filtration system such as a hollow fiber element. The analyzer including the separation system, the transfer element, and the movement system may at least partially automate a process of analyzing a blood sample in a tube.

According to an eight aspect there is provided an analyzer system. The analyzer system includes a cassette and an analyzer. The analyzer is configured to receive a tube containing a blood sample and the cassette. The analyzer includes a separation system, a transfer element, a movement system, and a cassette receptacle. The transfer element is configured to obtain at least portion of the blood sample from the tube. The transfer element is configured to deposit the portion of the blood sample into the separation system. The movement system is configured to move the transfer element between the tube and the separation system. The cassette receptacle is defined by the separation system. The cassette receptacle is configured to removably receive the cassette therein. The separation system is configured to separate the portion of the blood sample into a separated sample via the cassette. The separated sample includes one of a plasma sample and a serum sample.

According to a ninth aspect there is provided a method of analyzing a blood sample in a tube. The method includes placing the tube into an analyzer. The method further includes transferring at least a portion of the blood sample from the tube into a separation system of the analyzer. The transferring is performed automatically within the analyzer. The method further includes separating the portion of the blood sample into a separated sample by the separation system. The separated sample is one of a plasma sample and a serum sample. The method further includes transferring the separated sample into an analysis system. The transferring is performed automatically within the analyzer. The method further includes analyzing the separated sample via the analysis system.

According to a tenth aspect, there is provided a cassette. The cassette configured to be selectively disposed in an analyzer. The cassette includes a main body, at least one separation channel disposed within the main body. Each separation channel is configured to separate at least a portion of a blood sample into a separated sample.

According to an eleventh aspect, there is provided an analyzer. The analyzer is configured to receive a tube containing a blood sample. The analyzer includes a separation system. The separation system includes a filtration system. The filtration system includes a hollow fiber element, a transfer element, and a movement system. The transfer element is configured to obtain at least a portion of the blood sample from the tube. The transfer element is configured to deposit the portion of the blood sample into the separation system. The movement system is configured to move the transfer element between the tube and the separation system. The separation system is configured to separate the portion of the blood sample into a separated sample. The sample includes one of a plasma sample and a serum sample.

According to a twelfth aspect, there is provided a method of analyzing a blood sample in a tube. The method includes placing the tube into an analyzer. The method further includes transferring at least a portion of the blood sample from the tube into a separation system of the analyzer. The transferring is performed automatically within the analyzer. The separation system includes a filtration system. The filtration system includes a hollow fiber element. The method further includes separating the portion of the blood sample into a separated sample by the separation system. The separated sample is one of a plasma sample and a serum sample. The method further includes transferring the separated sample into an analysis system. The transferring is performed automatically within the analyzer. The method further includes analyzing the separated sample via the analysis system.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a diagrammatic view of an exemplary system for blood sample collection and processing;

FIG. 2 depicts an elevational view of an exemplary smart tube system of the system for blood sample collection and processing of FIG. 1;

FIG. 3 depicts a diagrammatic view of an exemplary computer architecture for use in the system for blood sample collection and processing of FIG. 1;

FIG. 4 depicts an elevational view of an exemplary tube of the smart tube system of FIG. 2;

FIG. 5 depicts an elevational view of an exemplary holder of the smart tube system of FIG. 2;

FIG. 6A depicts an exemplary filtration system incorporating a hollow fiber element for use in filtering plasma from whole blood;

FIG.6B depicts a cross-sectional view of the hollow fiber element of FIG. 6A taken along line 133-133;

FIG. 6C depicts an enlarged view of a portion of FIG. 6B;

FIG. 7 depicts a flowchart of an exemplary method for blood sample collection and processing;

FIG. 8 depicts a perspective view of an exemplary device for blood collection and separation;

FIG. 9 depicts a perspective view of the device of FIG. 8 with parts cut away;

FIG. 10 depicts a cross-sectional view of an exemplary rack;

FIG. 11 depicts a perspective view of the device of FIG. 8 with parts cut away and with an exemplary syringe element incorporated therewith;

FIG. 12 depicts a side elevational view of another exemplary rack with the device of FIG. 8 disposed therein;

FIG. 13 depicts a flowchart of an exemplary method for collecting and separating a blood sample;

FIG. 14 depicts a diagrammatical view of an exemplary analyzer;

FIG. 15 depicts a diagrammatical view of an exemplary cassette for use in the analyzer of FIG. 14;

FIGS. 16A-16B depict a perspective view and a top plan view, respectively, of an exemplary integrated handler element for use in plasma generation in the analyzer of FIG. 14;

FIGS. 17A-17C depict a perspective view, a top plan view, and a side elevational view, respectively, of an exemplary integrated wheel element for use in plasma generation in the analyzer of FIG. 14;

FIG. 18 depicts a perspective view of an exemplary portion of the analyzer of FIG. 14 along with an exemplary rack with an exemplary sample collection tube disposed therein;

FIG. 19 depicts a side elevational view of an exemplary sample collection tube with an exemplary insert disposed therein;

FIG. 20 depicts a diagrammatical view of a flowchart of an exemplary method of using the analyzer of FIG. 14;

FIGS. 21A-21B depict various perspective views of exemplary hollow fiber plasma separation modules for use in an exemplary analyzer system;

FIGS. 21C-21D depict various cross-sectional views of exemplary racks for use in an exemplary analyzer system;

FIGS. 22A-22B depict a perspective view and a side elevation view, respectively, of an exemplary movement system and exemplary loading areas for use in an exemplary analyzer system;

FIGS. 23A-23B depict top plan views of the movement system and loading areas of FIGS. 22A-22B;

FIGS. 24A-24B depict perspective views of a top of an exemplary hollow fiber plasma separation module similar to the one shown in FIGS. 21A-21B and an elevational view of an exemplary hollow fiber plasma separation module similar to the one shown in FIGS. 21A-21B for use in an exemplary analyzer system;

FIG. 25 depicts a side profile view of the hollow fiber plasma separation module of FIGS. 24A-24B disposed in an exemplary rack for use in an exemplary analyzer system;

FIG. 26A depicts a perspective view of an exemplary plasma separation rack for use in an exemplary analyzer system; and

FIG. 26B depicts a perspective view of an exemplary sample cuvette for use in an exemplary analyzer system.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Blood Sample Collection and Processing System

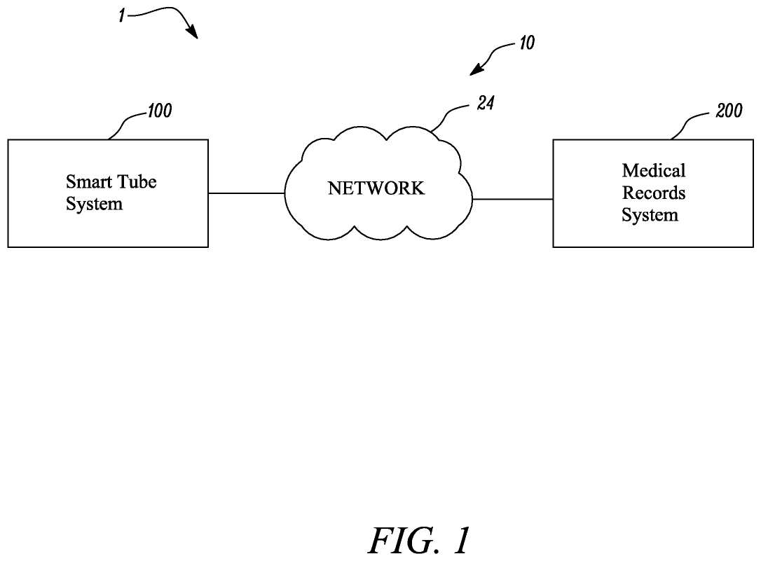

Referring now to FIG. 1, an exemplary blood sample collection and processing system (1) of the present invention may include an operating environment (10). In general, blood sample collection and processing system (1) operates to pre-analyze a whole blood sample for sufficiency prior to the full biochemical analysis. The pre-analysis is conducted in a rapid manner to provide a sufficiency determination to the healthcare professional regarding the blood sample. For example, a determination may be made while the patient is in the examination room or otherwise still present or in close proximity. If the sample is determined to be insufficient, the healthcare professional can counsel the patient regarding the proper fasting or other preparation techniques, redraw another whole blood sample, or examine the patient further.

Blood sample collection and processing system (1) may be applied for testing in locations such as triaging at patient home via emergency medical technicians, to decide if an ambulance and/or hospitalization is required. In ambulance testing may be performed by generating plasma for immediate testing in the ambulance. Communication systems and/or information technology systems may be incorporated with blood sample collection and processing system (1) to allow information and test results to be communicated to the emergency room, hospital, or care provider prior to the arrival of the patient. A virtual doctor could also be used in conjunction with blood sample collection and processing system (1).

In other environments and applications, blood sample collection and processing system (1) may be used in self-testing to prevent impaired operation of machinery such as cars, aircraft, or manufacturing. Other applications may relate to bar testing to prevent liability to the bar; athlete testing to identify doping; rehabilitation centers to monitor patients; prison and probation situations to monitor inmates; schools to monitor teachers and students; drug testing for employment and compliance; drug interactions with drugs of abuse (“DOA”) as current DOA testing is done based on urine, breath, saliva, and/or hair analysis; horse, dog, and/or camel racing and other doping opportunities; home testing for teens or other high risk groups; welfare recipients; jury selection; automatic safety system for vehicle lockout; and/or truck driver monitoring.

In addition to human blood testing, there is a need to test animal blood in a variety of settings. For example, there is a need to test animal blood in veterinarian offices; in large animal clinics; in small animal clinics; in animal production facilities such as chicken farms, in food production, in meat/food quality testing; in zoos; in nature preserves for wild animals; for tagged animals; in U.S.D.A. facilities and/or government animal regulatory facilities; in ports of entry for disease testing; in quarantine; in animal health monitoring for antibody production; in animal health monitoring for diagnostic controls; in research, university, and/or industry space; in vaccine testing; in cosmetic testing; in drug and/or pharmaceutical testing; in testing for rabies; in animal shelters; in pet stores; in grooming pet spas such as for a quick pet check during a grooming session. Providing a quick way to initiate a biochemical analysis such as through blood sample collection and processing system (1) may provide remote access to testing at farms, wildlife refuges, zoos, or other environments. A simple method for biochemical testing such as via blood sample collection and processing system (1) allows lower skilled personnel to conduct the testing. Current testing for animals is done by sending samples out to large reference labs or large veterinarian clinics. A faster turnaround time such as the turnaround time provided by blood sample collection and processing system (1) may increase test usage and provide faster results for emotional pet owners. Blood sample collection and processing system (1) may aid in standardizing hematocrit in animals. Animals with very little blood such as avian animals may be processed via blood sample collection and processing system (1) due to the need for less volume of whole blood.

With respect to field testing for health monitoring, fatigue testing, and dosing, blood sample collection and processing system (1) may provide accurate measurements quickly for both monitoring and trauma, such as for soldiers; sailors; endurance athletes; astronauts; truck drivers; machine operators; pilots; and diabetic, thyroid, and/or allergy patients. Areas of low resources such as remote areas or in disaster response environments may benefit from in-field testing using blood sample collection and processing system (1).

The ability to quickly generate plasma without large, expensive, and/or complicated instrumentation such as centrifugation may be addressed by the present disclosure. For certain tests, plasma testing is often likely to give a more accurate and more sensitive result than whole blood testing. The blood sample collection and processing system (1) may also enable more sensitive and/or specific solution-based testing when compared to current practice.

The amount of plasma needed for a given test based on test orders may be calculated and incorporated into the whole blood draw requirements. A phlebotomist may be guided to the right type, number, and tube size for the sample draws. The sample stability (aging) and sample quality may be monitored by blood sample collection and processing system (1). The sample quality may also be provided to a central analysis platform for tracking, tracing, and auditing the plasma generation system or any other components of blood sample collection and processing system (1).

The blood collection and processing may be provided through a smart tube system (100) and a medical records system (200). In some versions of operating environment (10), smart tube system (100) and medical records system (200) may send and receive communications data between one another directly. Alternatively, in other versions of operating environment (10), smart tube system (100) and medical records system (200) may communicate with each other through a network (24). Network (24) may include one or more private or public networks (e.g., the Internet) that enable the exchange of data. In yet other versions of operating environment (10), medical records system (200) is omitted.

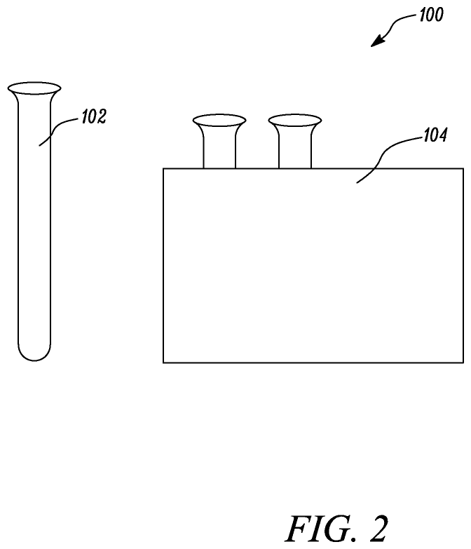

As shown in FIG. 2, smart tube system (100) includes a tube (102) and a holder (104). In general, tube (102) is configured to receive a whole blood sample therein and generate a plasma sample from the blood sample within tube (102). The plasma sample is generated in a rapid manner without using centrifugation and may be accomplished with a small whole blood sample. For example, in some versions of smart tube system (100), a plasma sample may be generated from less than 300 microliters (μL) of whole blood.

Once tube (102) contains a whole blood sample, tube (102) is thereafter placed in holder (104) for analysis of the blood sample and/or plasma sample. The medical records of the patient associated with the blood sample may be accessed through medical records system (200) by holder (104) to aid in analyzing the plasma sample or for use in labeling tube (102). Conversely, holder (104) may provide information regarding the patient's plasma sample to medical records system (200) for storage therein.

A. Exemplary Computer System

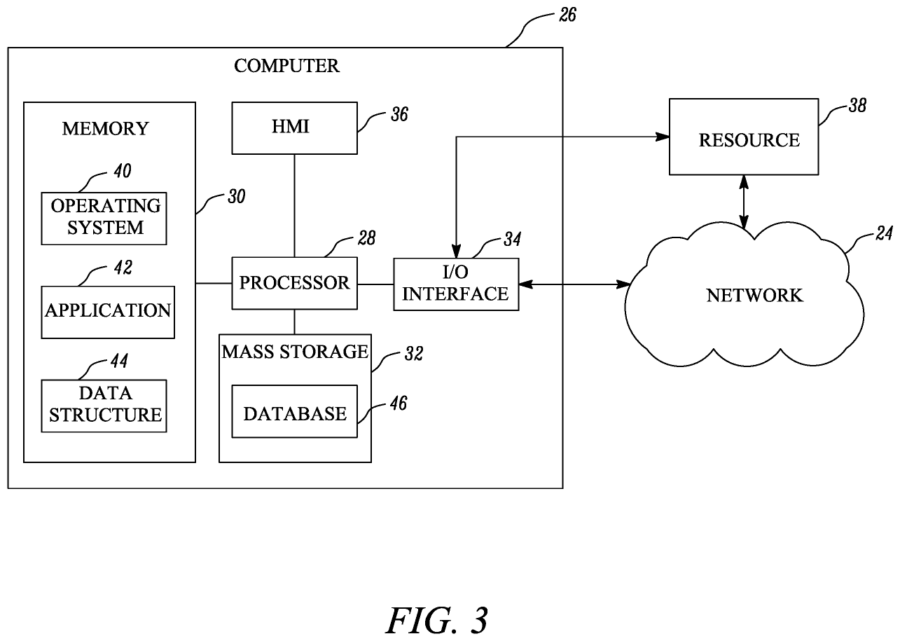

Referring now to FIG. 3, all or portions of smart tube system (100), medical records system (200), and network (24) of operating environment (10) may be implemented on one or more computing devices or systems, such as an exemplary computer system (26). Computer system (26) may include a processor (28), a memory (30), a mass storage memory device (32), an input/output (I/O) interface (34), and a Human Machine Interface (HMI) (36). Computer system (26) may also be operatively coupled to one or more external resources (38) via network (24) or I/O interface (34). External resources may include, but are not limited to, servers, databases, mass storage devices, peripheral devices, cloud-based network services, or any other suitable computer resource that may be used by computer system (26).

Processor (28) may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory (30). Memory (30) may include a single memory device or a plurality of memory devices including, but not limited, to read-only memory (ROM), random access memory (RAM), volatile memory, non-volatile memory, static random-access memory (SRAM), dynamic random access memory (DRAM), flash memory, cache memory, or any other device capable of storing information. Mass storage memory device (32) may include data storage devices such as a hard drive, optical drive, tape drive, non-volatile solid-state device, or any other device capable of storing information.

Processor (28) may operate under the control of an operating system (40) that resides in memory (30). Operating system (40) may manage computer resources so that computer program code embodied as one or more computer software applications, such as an application (42) residing in memory (30), may have instructions executed by processor (28). In an alternative embodiment, processor (28) may execute the application (42) directly, in which case operating system (40) may be omitted. One or more data structures (44) may also reside in memory (30), and may be used by processor (28), operating system (40), or application (42) to store or manipulate data.

I/O interface (34) may provide a machine interface that operatively couples processor (28) to other devices and systems, such as network (24) or external resource (38). Application (42) may thereby work cooperatively with network (24) or external resource (38) by communicating via I/O interface (34) to provide the various features, functions, applications, processes, or modules comprising embodiments of the invention. Application (42) may also have program code that is executed by one or more external resources (38), or otherwise rely on functions or signals provided by other system or network components external to computer system (26). Indeed, given the nearly endless hardware and software configurations possible, persons having ordinary skill in the art will understand that embodiments of the invention may include applications that are located externally to computer system (26), distributed among multiple computers or other external resources (38), or provided by computing resources (hardware and software) that are provided as a service over network (24), such as a cloud computing service.

HMI (36) may be operatively coupled to processor (28) of computer system (26) in a known manner to allow a user to interact directly with computer system (26). HMI (36) may include video or alphanumeric displays, a touch screen, a speaker, and any other suitable audio and visual indicators capable of providing data to the user. HMI (36) may also include input devices and controls such as an alphanumeric keyboard, a pointing device, keypads, pushbuttons, control knobs, microphones, etc., capable of accepting commands or input from the user and transmitting the entered input to processor (28).

A database (46) may reside on mass storage memory device (32), and may be used to collect and organize data used by the various systems and modules described herein. Database (46) may include data and supporting data structures that store and organize the data. In particular, database (46) may be arranged with any database organization or structure including, but not limited to, a relational database, a hierarchical database, a network database, or combinations thereof. A database management system in the form of a computer software application executing as instructions on processor (28) may be used to access the information or data stored in records of database (46) in response to a query, where a query may be dynamically determined and executed by operating system (40), other applications (42), or one or more modules.

B. Exemplary Tube

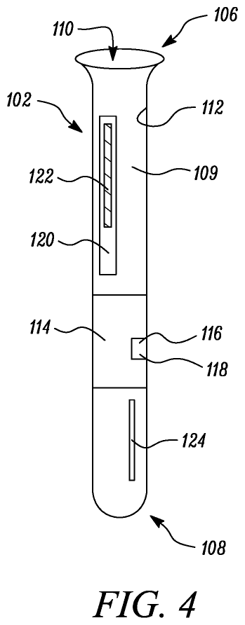

As shown in FIG. 4, tube (102) extends from a top end (106) to a bottom end (108) and includes an exterior surface (109). A top opening (110) is defined at top end (106). Top opening (110) opens to a collection chamber (112) defined within tube (102) to allow for the blood sample to be placed inside tube (102).

A plasma separation system (114) is disposed within collection chamber (112). Plasma separation system (114) is configured to filter, convert, or otherwise extract a portion of the blood sample disposed in collection chamber (112) into a plasma sample without using centrifugation. In some versions of tube (102), plasma separation system (114) may comprise a filtration system incorporating a hollow fiber element. Other versions of tube (102) may incorporate various features from hollow fiber element into plasma separation system (114).

Plasma separation system (114) acts to filter out or otherwise extract plasma from the whole blood placed into collection chamber (112). In some versions of plasma separation system (114), the non-plasma elements of the filtered whole blood sample travels through plasma separation system (114) and is deposited proximate bottom end (108) of tube (102), while the plasma sample is maintained within plasma separation system (114). Plasma separation system (114) may be configured to separate the plasma sample from the whole blood sample in a rapid manner. In some versions of plasma separation system (114), the plasma sample may be generated in under ten minutes.

Collection chamber (112) may define an analysis pocket (116). Analysis pocket (116) operates cooperatively with plasma separation system (114) to receive a unit of the plasma sample therein. Tube (102) includes a portion (118) adjacent to analysis pocket (116). In some versions of tube (102), portion (118) is transparent to allow for sufficient optical imaging of the unit of the plasma sample therein.

On exterior surface (109) of tube (102), a label portion (120) may be defined and configured to receive a label (122) thereon. Exterior surface (109) of label portion (120) may include a different physical property from the rest of exterior surface (109). For example, exterior surface (109) of label portion (120) may include a textured surface to aid in receiving and adhering label (122) thereon. Exterior surface (109) of label portion (120) may be a flattened or planer area, rather than the typical angular or rounded surface on other portions of tube (102) to aid in receiving label (122). Label (122) may alternatively comprise an electronic label such as an RFID chip programmable to include patient information or other relevant information therein.

Alternatively, labeling may be applied directly to the outer surface of tube (102) by etching or otherwise imprinting onto tube (102). In another example, a pre-applied substrate already on tube (102) may be imprinted upon.

In some versions of tube (102), an alignment element (124) may extend from exterior surface (109). Alignment element (124) may be a fin, protuberance, flange or an otherwise “male” style element for aligning tube (102) in a particular orientation within a corresponding “female” style element of holder (104). Conversely, alignment element (124) may be a female style element, while holder (104) may provide the corresponding male element. In other versions of tube (102), alignment element may be another style of mechanism for positioning tube (102) in a desired orientation within holder (104). For example, a magnetic connection may be provided to align tube (102) or any other commonly used features for positioning tube (102) in a particular orientation with respect to holder (104).

C. Exemplary Holder

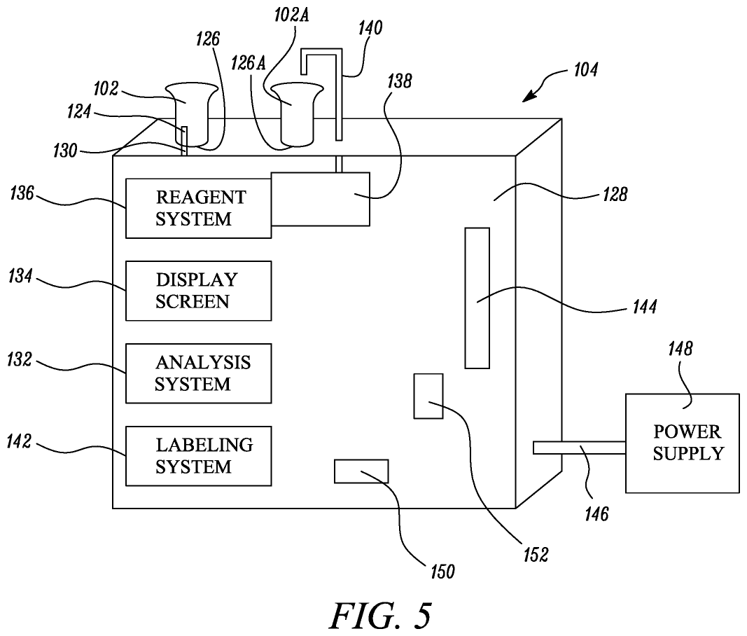

As shown in FIG. 5, holder (104) is configured to receive tube (102) in a tube receptacle (126 defined by a body (128) of holder (104). An alignment element (130) is associated with tube receptacle (126) and configured to cooperate with alignment element (124) of tube (102) to position tube (102) in a particular orientation within holder (104). In some versions of blood sample collection and processing system (1), alignment element (124) of tube (102) is one of a male or female element and alignment element (130) of holder (104) is the other one of the male or female element. For example, alignment element (124) may be a fin extending from exterior surface (109) of tube (102), while alignment element (130) may be a recess or notch associated with tube receptacle (126) and sized to receive the fin of alignment element (124) therein.

Holder (104) may be configured to receive multiple tube elements therein. For example, tube (102) and tube (102A) may both be disposed within a corresponding tube receptacle (126) and tube receptacle (126A), respectively. Different versions of holder (104) may be configured to receive different numbers of tubes (102).

Some versions of holder (104) include an analysis system (132) configured to collect a set of data from the unit of plasma disposed in analysis pocket (116) once tube (102) is disposed in holder (104). Analysis system (132) may include various optical features such as a spectroscopic element for conducting an optical analysis of the unit of plasma. Analysis system (132) is configured to rapidly analyze the whole blood and/or unit of plasma. The rapid analysis may take place while the patient is still in the examination room or otherwise still present or in close proximity to allow immediate action if sample quality is poor. With the patient still in the examination room or otherwise still present or in close proximity, the healthcare professional can either redraw a sample of blood from the patient, counsel the patient regarding the issues with the blood sample, or examine the patient further.

For example, analysis system (132) may determine the quality of plasma to ensure acceptable levels of interferents such as hemoglobin, lipids, or bilirubin. If unacceptable levels of these interferents are present, the healthcare professional may counsel the patient, who is still in the examination room or otherwise still present or in close proximity, regarding the necessary steps to take to ensure the next sample is acceptable (e.g., fast longer, refrain from eating fatty foods prior to the blood draw, etc.), redraw the sample of blood, or examine the patient further. Analysis system (132) may generate a capillary serum index for use in determining whether the whole blood sample is sufficient. Methods for generating a capillary serum index are disclosed in U.S. Pat. Nos. 9,007,574 and 9,588,038 and are incorporated herein by reference.

Some versions of holder (104) include a display screen (134). Display screen (134) may provide a graphical user interface similar to I/O interface (34) as described above. In some versions of holder (104), display screen (134) is actuated by analysis system (132) to display information related to the set of data collected from the unit of plasma disposed in analysis pocket (116) and analyzed by analysis system (132). This information may include hemoglobin, lipid, or bilirubin levels.

In as much as analysis system (132) is configured to provide a rapid analysis of the unit of plasma, in some scenarios, the patient is still in the examination room or otherwise still present or in close proximity when the analysis is completed. In response to reviewing the information, the healthcare professional may determine the sample of whole blood is insufficient for a particular purpose and counsel or advise the patient accordingly. For example, analysis of the unit of plasma via analysis system (132) may indicate poor quality plasma, with unacceptable levels of interferents such as hemoglobin, lipids, or bilirubin.

In some versions, holder (104) includes a reagent system (136). Reagent system (136) includes a reservoir (138) filled with one or more reagents for use in mixing with the whole blood sample or the plasma sample to isolate or indicate the presence or absence of a particular element. Reagent system (136) includes corresponding deposit element (140) comprised of tubing, channels, and/or a nozzle extending from body (128) of holder (104) for use in delivering the selected reagent into collection chamber (112) of tube (102) once tube (102) is disposed in holder (104).

For example, the healthcare professional may desire to determine an amount of a particular protein in a whole blood sample. After placing tube (102) into holder (104), the healthcare professional actuates an interface on display screen (134) to actuate reagent system (136) to transfer a deposit of “Reagent A” from reservoir (138), through deposit element (140) and into collection chamber (112) of tube (102). Once Reagent A is mixed with the whole blood sample, analysis system (132) determines the amount of a particular protein in the whole blood sample and provides this information on display screen (134) for review by the healthcare professional.

In some versions, holder (104) includes a labeling system (142). Labeling system (142) is configured to apply label (122) to label area (120) of tube (102). In some versions of blood sample collection and processing system (1), smart tube system (100) receives information regarding the patient from medical records system (200) and applies this information to label (122). For example, identification information for the patient may be received from medical records system (200), formatted, and applied to label (122) by labeling system (142). Thereafter, tube (102) includes the patient identification system. In some versions of labeling system (142), information may be converted into a machine-readable format such as a bar code or QR code for application onto label (122).

Labeling system (142) may incorporate data supplied by the healthcare professional through display screen (134) into the information printed on label (122). The patient's contact information, date of examination, the healthcare professional's initials, or any other information may be entered by the healthcare professional into display screen (134) and transferred onto label (122). Labeling system (142) may include a laser or inkjet style printing element for transferring information onto label (122). In other versions of labeling system (142), the information is printed or transferred directly onto tube (102) forgoing label (122). This may be achieved by etching or otherwise imprinting the information onto label area (120) directly. In other versions of labeling system (142), label (122) incorporates a radio-frequency identification tag technology into the physical body of label (122) to allow for location tracking and passive information transmissions from label (122) regarding tube (102).

In those versions of label (122) embodied in an electronic label such as an RFID chip, labeling system (142) is configured to program label (122) with patient data or other relevant information.

Current labeling solutions rely on a healthcare professional manually transferring a handwritten or computer-generated label from onto a tube. Mislabeled samples lead to incorrect testing, incorrect diagnosis, and incorrect treatment. Studies indicate the financial effect of a mislabeled sample is hundreds of dollars and the emotional impact is incalculable. Further, correct placement of a label onto a tube is important for sample collection. A wrinkled, twisted, non-horizontal, incorrectly oriented label, or a label which prevents observation of the sample can cause delays in processing the sample or may even require the patient to provide another whole blood sample. Labeling system (142) reduces sample mislabeling by simplifying the workflow for the healthcare professional through automatic application of a label to the corresponding tube (102) in a correct and repeatable placement and orientation.

Power may be provided to holder (104) through a battery (144) or via a power cord (146) connected to a power supply (148). In some versions of holder (104), power supply (148) is used for both charging battery (144) as well as supplying holder (104) with power. In other versions, battery (144) is charged directly through power supply (148) and holder (104) runs off battery (144). Battery (144) may be swappable with another battery (144).

As shown in FIG. 5, communication between holder (104) and medical records system (200) may be facilitated through a wired network connection formed through network jack (150). Network jack (150) is configured to receive one end of a communication cable and may be an “ethernet” style or any other style jack for facilitating wired communications with network (24). Communication between holder (104) and medical records system (200) may be facilitated through a wireless network connection formed through wireless module (152). Wireless module (152) is configured to wirelessly communicate with a corresponding wireless module such as those found in wireless modems for facilitating wireless communication with network (24).

D. Exemplary Plasma Filtration System

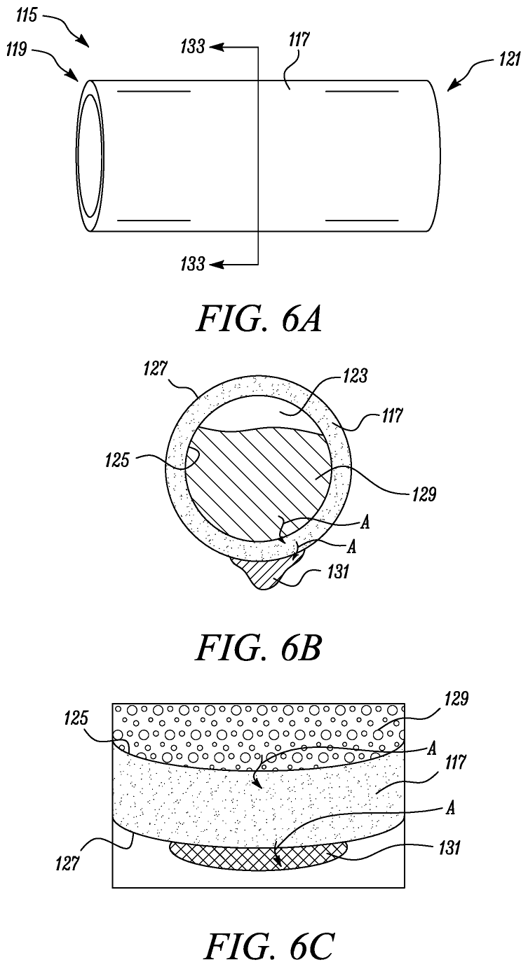

As shown in FIG. 6A, plasma separation system (114) may comprise a plasma filtration system incorporating a hollow fiber element (115). In general, hollow fiber element (115) receives as input whole blood and filters out plasma from the whole blood. In some versions of hollow fiber element (115), the walls of hollow fiber element (115) allow plasma to pass therethrough but retain or restrict the remainder of the whole blood from passing therethrough. Thus, hollow fiber element (115) separates the plasma from the whole blood for use as needed by smart tube system (100).

In the version of hollow fiber element (115) depicted in FIGS. 6A-6C, hollow fiber element (115) is generally comprised of a filter wall (117) extending from a first end (119) to a second end (121) and defining an interior (123) therein. Filter wall (117) is depicted as tubular, though any shape or orientation of filter wall (117) is contemplated. Fluid, such as whole blood, may be disposed in interior (123). Filter wall (117) includes an interior surface (125) and an exterior surface (127). In general, filter wall (117) is comprised of one or more materials sufficient to allow plasma from the whole blood to move through filter wall (117), while retaining non-plasma material of the whole blood within interior (123). Materials such as ceramic and various coatings may be incorporated into filter wall (117) to facilitate the transfer of plasma therethrough while preventing the transfer of non-plasma therethrough.

In operation, a whole blood sample (129) is disposed within interior (123). Filter wall (117) is comprised of one or more materials configured to allow plasma to seep or otherwise move through filter wall (117), while simultaneously preventing the seeping or movement of non-plasma materials such as red and white blood celled through filter wall (117). This movement is shown with respect to Arrow A in FIGS. 6B and 6C, with a sample of plasma (131) disposed on exterior surface (127) after moving through filter wall (117) from interior (123). Interior surface (125), exterior surface (127), or both may be coated with one or more coating or resins that aid in preventing non-plasma from passing therethrough. A pressure system (not shown) may also be employed to pressurize interior (123) to speed up or increase the transfer of plasma through filter wall (117) and drive the fluid through the hollow fiber.

The plasma is thereafter collected from exterior surface (127) and the general exterior of hollow fiber element (115), effectively separating the plasma from the rest of the whole blood. This allows smart tube system (100) to quickly produce plasma for further use therein without centrifugation.

II. Exemplary Blood Sample Collection and Processing Method

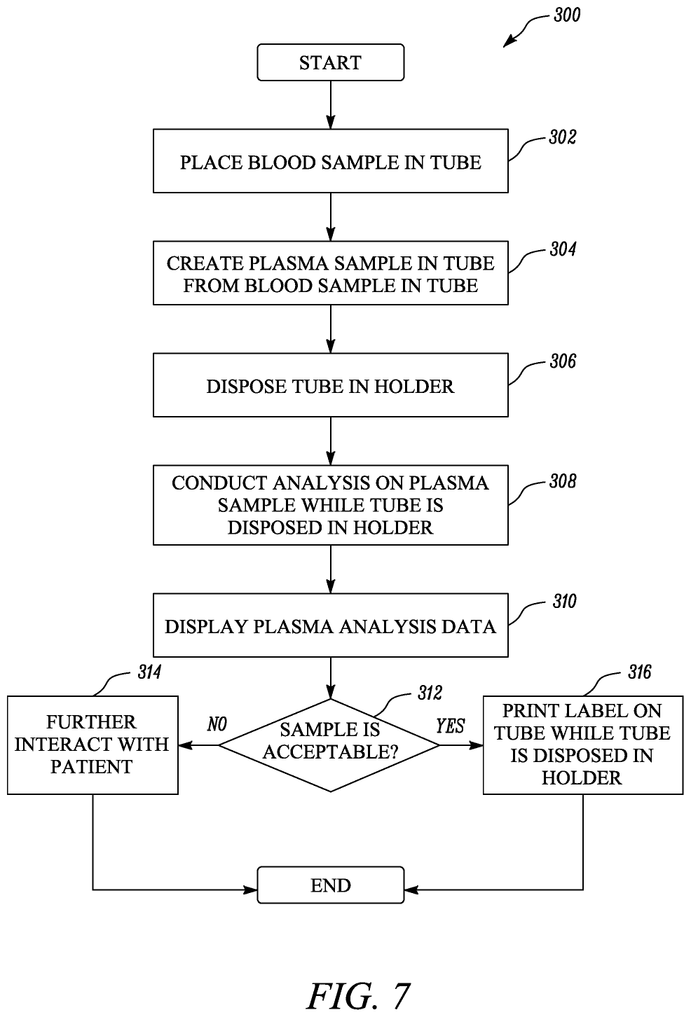

As shown in FIG. 7, a method (300) may be used to collect and process a blood sample. Method (300) begins with a step (302), whereby a blood sample is collected from a patient and placed in a tube. The tube of method (300) may be similar to tube (102) as described above. The blood sample may be withdrawn from the patient directly into the tube, or indirectly withdrawn into an intermediate container and thereafter disposed into the tube. Thereafter, step (302) proceeds to a step (304).

In step (304), a plasma sample is created in the tube from the blood sample disposed in the tube. The mechanism for creating the plasma sample may be in the form of a filtration system such as those incorporating a hollow fiber element or a similar system for creating a plasma sample from the blood sample within the tube. Hollow fiber element may incorporate a filter wall or other material for filtering plasma out of whole blood, thus producing plasma from whole blood without centrifugation. Thereafter, step (304) proceeds to a step (306). In step (306), the user disposes the tube in a holder. The holder of method (300) may be similar to holder (104) as described above. While method (300) is shown with step (304) proceeding step (306), in other versions of method (300), step (306) may proceed step (304), with the user placing the tube into the holder and thereafter the plasma sample created from the blood sample. After the tube is disposed in the holder, step (306) moves to a step (308).

In step (308), the plasma sample in the tube is analyzed by the holder. In some versions of the holder, an analysis system of the holder may be configured to analyze the plasma sample in the tube. The analysis system of method (300) may be similar to analysis system (132) as described above. The analysis system may optically analyze the plasma in the tube to conduct the analysis and to determine whether the plasma sample and/or the blood sample is sufficient or whether an action such as drawing another blood sample is necessary. Inasmuch as the analysis is conducted rapidly, the patient is still in the examination room or otherwise still present or in close proximity and readily available for another blood draw or in-person counseling by the healthcare professional. After the plasma has been analyzed, step (308) proceeds to a step (310).

In step (310), the data collected in step (308) regarding the plasma sample is displayed on a display screen to the healthcare professional. The display screen of method (300) may be similar to display screen (134) as discussed above. Thereafter, step (310) moves to a step (312), whereby the healthcare professional reviews the data and determines whether the blood sample is acceptable. If the blood sample is not acceptable, step (312) moves to a step (314), where the healthcare professional initiates further interaction with the patient. Thereafter, method (300) ends. If the blood sample is acceptable, step (312) moves to a step (316).

In step (316), a labeling system of holder prints information onto a label and affixes the label onto the tube. Labeling system and label are similar to labeling system (142) and label (122), respectively, as discussed above. The holder may acquire patient information or other data via a communication port connected to a medical records system and pass this information to the labeling system for printing of the label. In some versions of step (316), the labeling system receives patient data from the medical records system and prints this information onto the label. The label is then affixed to the tube to allow for handling and movement of the tube with proper identification. Thereafter, method (300) ends.

III. Exemplary Device for Blood Collection and Separation

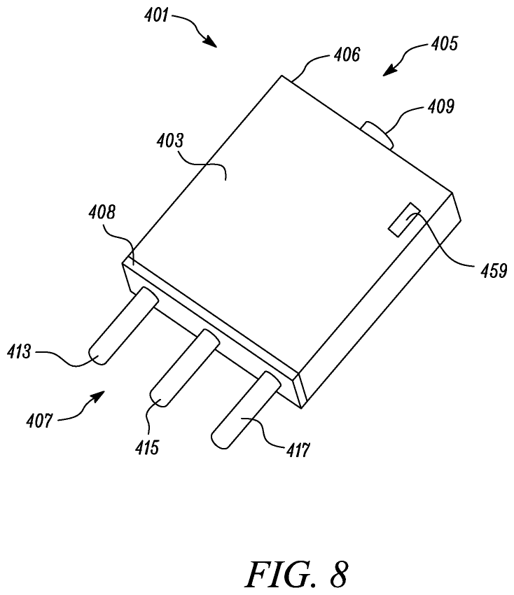

Referring now to FIG. 8, an exemplary device for blood collection and separation of the present invention is depicted as a device (401). Device (401) is configured to receive a needle and be inserted into the vein of a patient to draw a blood sample. Device (401) is configured to both draw and separate the blood sample. During blood draw, device (401) may separate the blood sample into separate materials such as whole blood, plasma, and/or serum and move these separated materials into isolated chambers. The blood separation technology may use different methods for separating sample types. For example, filters, anticoagulants, resins, micro-channels, special coatings or similar may be used. In some versions of device (401), one chamber may have an EDTA coating for whole blood separation, one chamber may have a plasma filter and EDTA for plasma separation, and one chamber may have spray silica coating for serum separation. Thereafter, device (401) may be loaded directly onto a rack of an analyzer instrument for processing. Alternatively, the individual containers of separated material may be removed from device (401) and loaded into a rack of the analyzer instrument. Portions of device (401) may include an identification element such as a bar code, color code, or RFID tag to provide information to the analyzer instrument.

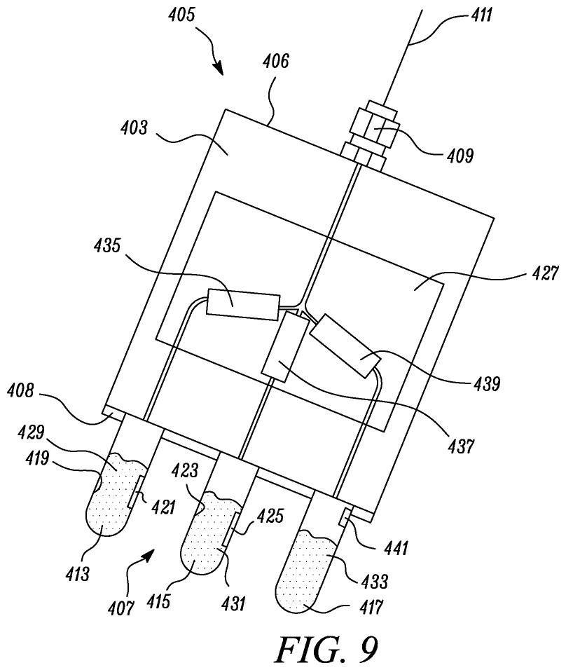

Device (401) includes a main body (403) extending from a front end (405) to a back end (407) and having a front wall (406) disposed at front end (405) and a back wall (408) disposed at back end (407). Device (401) includes a needle attachment feature (409) disposed at front end (405) and extending from front wall (406). Needle attachment feature (409) is configured to couple a needle element (411) (shown in FIG. 9) to main body (403). Further, needle element (411) is configured to transfer a blood sample from needle element (411) through front wall (406) and into main body (403). Needle element (411) may comprise a blood draw needle, a finger stick needle, or any other mechanism for drawing a blood sample from a patient.

As shown in FIGS. 8 and 9, multiple chambers are disposed at back end (407) of main body (403). In some versions of device (401), a first chamber (413), a second chamber (415), and a third chamber (417) are disposed at back end (407) of main body (403). In other versions of device (401), less than three or more than three chambers are disposed at back end (407) of main body (403). In some versions of device (401), one or more of first chamber (413), second chamber (415), and/or third chamber (417) are removably secured to back wall (408). In some versions of device (401), one or more of first chamber (413), second chamber (415), and/or third chamber (417) comprise a test tube.

In some versions of device (401), one or more of first chamber (413), second chamber (415), and/or third chamber (417) include an interior surface, wherein the interior surface is coated with a layer of silica. For example, as shown in FIG. 9, first chamber (413) includes an interior surface (419) coated at least in part with a layer of silica (421). In some versions of device (401), one or more of first chamber (413), second chamber (415), and/or third chamber (417) include an interior surface, wherein the interior surface is coated with an anticoagulant. For example, as shown in FIG. 9, second chamber (415) includes an interior surface (423) coated at least in part with a layer of anticoagulant (425).

As shown in FIG. 9, device (401) includes a separation element (427) disposed within main body (403). Separation element (427) is configured to separate a blood sample acquired through needle element (411) into separate portions and thereafter transfer these portions into the chambers disposed at back end (407) of main body (403). In some versions of device (401), separation element (427) is configured to separate a blood sample into a first portion (429), a second portion (431), and a third portion (433). Thereafter, device (401) is configured to transfer first portion (429) into first chamber (413), second portion (431) into second chamber (415), and third portion (433) into third chamber (417).

In some versions of device (401), separation element (427) is configured to separate a blood sample into a sample of whole blood and deposit the sample of whole blood into one of the chambers. In some versions of device (401), separation element (427) is configured to separate a blood sample into a sample of plasma and deposit the sample of plasma into one of the chambers. In some versions of device (401), separation element (427) is configured to separate a blood sample into a sample of serum and deposit the sample of plasma into one of the chambers. Separation element (427) may comprise or include a plasma separation system (435), a micro-channel (437), a filter (439), an anticoagulant, or a resin for use in separating a blood sample into different portions.

As shown in FIGS. 6A-6C, plasma separation system (35) may comprise a plasma filtration system incorporating hollow fiber element (115). In general, hollow fiber element (115) receives as input whole blood and filters out plasma from the whole blood. In some versions of hollow fiber element (115), the walls of hollow fiber element (115) allow plasma to pass therethrough but retain or restrict the remainder of the whole blood from passing therethrough. Thus, hollow fiber element (115) separates the plasma from the whole blood for use as needed by device (401). The plasma is thereafter collected from exterior surface (127) and the general exterior of hollow fiber element (115), effectively separating the plasma from the rest of the whole blood. This allows device (401) to quickly produce plasma for further use therein without centrifugation. Details of hollow fiber element (115) has been discussed above with reference to FIGS. 6A-6C.

One or more of first chamber (413), second chamber (415), and/or third chamber (417) may include an identification element (441) such as a bar code, an RFID tag, or any other mechanism for identifying a specific chamber. Identification element (441) may be read by device (401) or another separate device for identifying a specific chamber. In some versions of device (401), identification element (441) is recognized by separation element (427) and separation element (427) is configured to separate the blood sample into a particular portion based at least in part on identification element (441). For example, first chamber (413) may include a particular identification element (441) associated with a request for a deposit of plasma therein. Separation element (427) recognizes the request for a deposit of plasma in first chamber (413) by identification element (441) and proceeds to separate a blood sample into a plasma portion and deposit this plasma portion into first chamber (413).

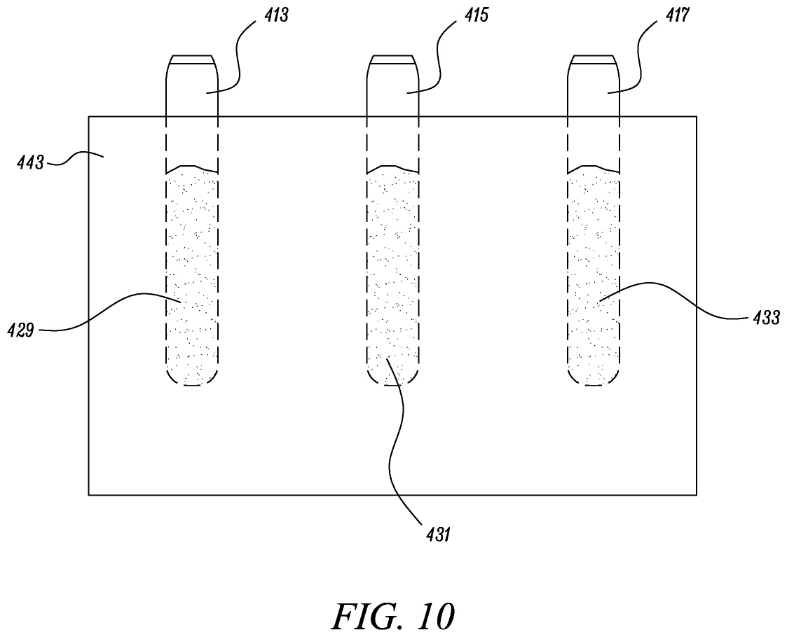

In some versions of device (401), one or more of first chamber (413), second chamber (415), and/or third chamber (417) are removably secured to back wall (408). Once the desired portions are separated out from a blood sample and transferred into first chamber (413), second chamber (415), and/or third chamber (417), the user may remove one or more of first chamber (413), second chamber (415), and/or third chamber (417) and deposit the removed chamber onto a rack (443) of an analyzer instrument (not shown), as shown in FIG. 10. Rack (443) or any other portion of the analyzer instrument may be configured to read any identification elements (441) of first chamber (413), second chamber (415), and/or third chamber (417) and to proceed with analyzing the particular chamber based on the associated identification element (441).

A vacuum is created within main body (403) to draw a blood sample from a patient once needle element (411) is connected to a patient's vein. As illustrated in FIG. 9, in some versions of device (401), needle attachment feature (409) cooperates with needle element (411) and main body (403) to utilize the internal vacuum when the user pushes main body (403) into needle element (411) and the blood is drawn automatically into main body (403).

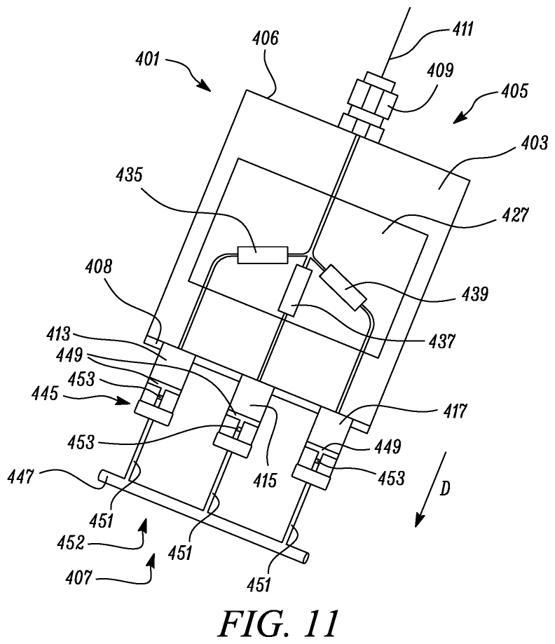

As illustrated in FIG. 11, in other versions of device (401), a syringe element (445) is coupled with first chamber (413), second chamber (415), and/or third chamber (417) and may be drawn away from back wall (408) to create the vacuum in main body (403). More specifically, syringe element (445) may include a handle (447), one or more stoppers (449), and a neck (451) extending between handle (447) and each stopper (449). The number of stoppers (449) correspond to the number of chambers. In the example illustrated in FIG. 11, three stoppers (449) are present, with first chamber (413), second chamber (415), and/or third chamber (417) each having one of stoppers (449) disposed therein. The user disposes each stopper (449) in the corresponding first chamber (413), second chamber (415), and/or third chamber (417) and when needle element (411) is connected to a patient's vein, pulls handle (447) in the direction of Arrow D (FIG. 11) to create a vacuum in main body (403) and draw a blood sample into separation element (427).

Once the user is done drawing a blood sample into main body (403), the user may disconnect and discard a disposable portion (452) of syringe element (445) to decrease the overall profile or size of device (401). To that end, some versions of syringe element (445) include a perforation or other type of breakaway feature on each neck (451) to facilitate removable of disposable portion (452), which includes handle (447) and at least a portion of neck (451). As shown in FIG. 11, a perforation (453) is provided on each neck (451). Once a blood sample is sufficiently drawn into main body (403), device (401) is removed from the patient. Thereafter, the user manipulates handle (447) to snap off disposable portion (452) from the remainder of syringe element (445) at each perforation (453). Each stopper (449) remains within one of first chamber (413), second chamber (415), and/or third chamber (417) to continue to cap the fluid within each chamber (413, 415, 417). The user may then discard disposable portion (452).

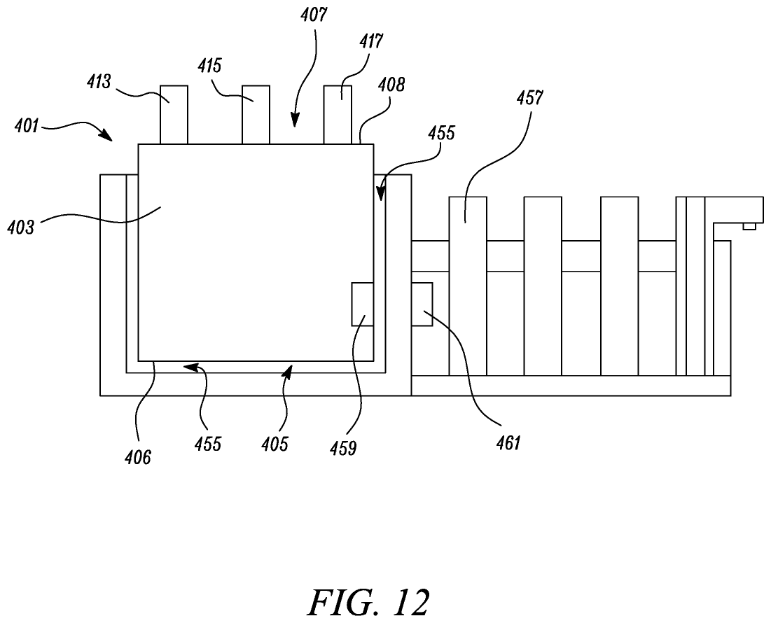

As illustrated in FIG. 12, in some versions of device (401), the user may omit removing first chamber (413), second chamber (415), and/or third chamber (417) from main body (403) and may place main body (403) directly into a device receptacle (455) of a rack (457) of an analyzer instrument (not shown). Rack (457) or any other part of analyzer instrument may be configured to detect the receipt of main body (403) therein and proceed with analyzing the material within first chamber (413), second chamber (415), and/or third chamber (417) accordingly. Main body (403) may include an identification element (459) comprising a bar code, an RFID tag, or any other mechanism for identifying a specific main body (403). Rack (457) may include a corresponding reader (461) for reading identification element (459) and using the information contained therein to determine how to analyze or process the material within first chamber (413), second chamber (415), and/or third chamber (417).

IV. Exemplary Methods for Blood Collection and Separation

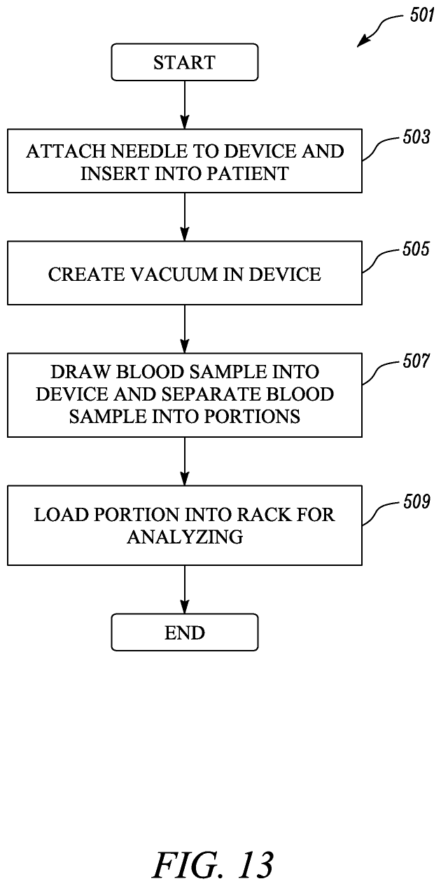

As shown in FIG. 13, various methods may be employed for blood collection and separation using a device similar to device (401) as described above. An exemplary method (501) begins with a step (503), whereby a needle element similar to needle element (411) is attached to a device similar to device (401) and inserted into a patient. Thereafter, step (503) proceeds to a step (505). In step (505), a vacuum is created in the device. The creation of a vacuum may be via any method common in the art. For example, a vacuum may be created by pressing on the device to expel air through a one-way valve. In another example, a syringe element similar to syringe element (445) may be incorporated into the device, whereby pulling on the syringe element creates a vacuum in the device. Once a vacuum is created in the device, step (505) moves to a step (507).

In step (507), a sample of blood is drawn from the patient through the needle element and into a main body of the device. Therein, the blood sample is separated into different portions. In some versions of step (507), the blood sample may be separated by a separation element similar to separation element (427). More specifically, in some versions of step (507), the blood sample may be separated into a whole blood portion, a plasma portion, and a serum portion and moved into isolated chambers such as first chamber (413), second chamber (415), and/or third chamber (417) as described above.

The mechanism for creating the plasma sample may be in the form of a filtration system such as those incorporating a hollow fiber element or a similar system for creating a plasma sample from the blood sample within the tube. Hollow fiber element may incorporate a filter wall or other material for filtering plasma out of whole blood, thus producing plasma from whole blood without centrifugation.

Thereafter, step (507) moves to a step (509). In step (509), the separated portions of the blood sample are loaded into a rack similar to either rack (443) or rack (457) above. In some versions of step (509), the portions are removed from the main body of the device and loaded directly into the rack. In other versions of step (509), the portions remain connected with the main body of the device and the main body is loaded into the rack for further processing. Once the separated portions of the blood sample are loaded into the rack, method (501) proceeds to end.

V. Exemplary Analyzer

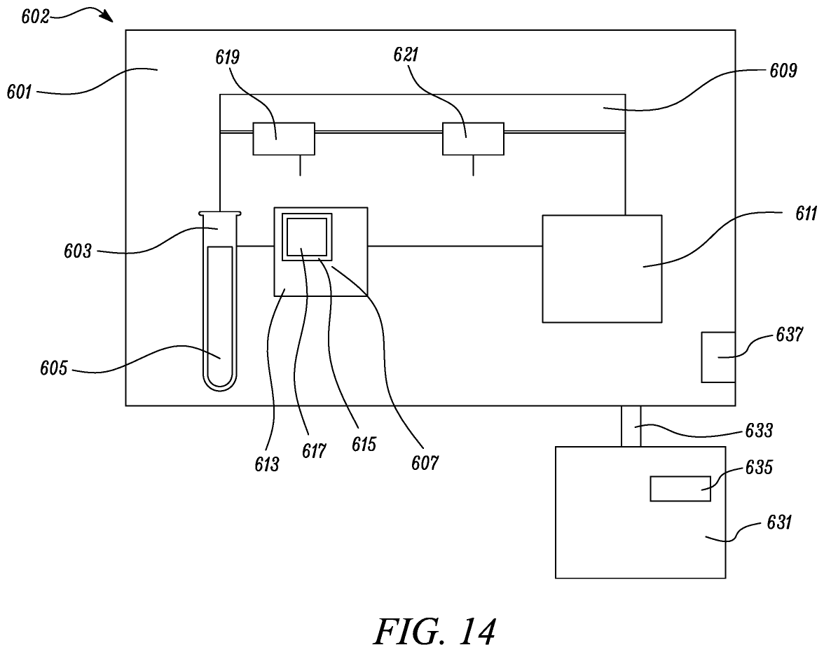

An exemplary analyzer (601) of the present invention is depicted in FIG. 14. Analyzer (601) is configured to receive a tube (603) containing a whole blood sample (605) therein. Analyzer (601) includes a separation system (607), a movement system (609), and an analysis system (611). Separation system (607) may be used to separate a whole blood sample into a separated sample. In some versions of analyzer (601), the separated sample may comprise either a plasma sample or a serum sample. Analysis system (611) is configured to conduct testing and analysis on the separated sample deposited therein. Analysis system (611) may comprise a chemistry analyzer, an immunoassay analyzer, a molecular analyzer, and/or a mass spectrometry analyzer.

Separation system (607) is configured to separate all or a portion of whole blood sample (605) into a separated sample such as a plasma sample or a serum sample. Separation system (607) may utilize or comprise any mechanism for converting whole blood sample (605) into a separated sample. For example, separation system (607) may include a filtration system. Some versions of the filtration system may comprise a hollow fiber system or otherwise use hollow fiber technology therein.

Analyzer (601) may be combined with a cassette (617) to form an analyzer system (602).

As such, some versions of separation system (607) include a main body (613) which defines a cassette receptacle (615). Cassette receptacle (615) is configured to removably receive cassette (617) therein.

Cassette (617) may be a consumable and disposable component of separation system (607) containing the mechanism for converting whole blood sample (605) into the separated sample. For example, cassette (617) may contain the above-mentioned elements relating to a filter system and/or hollow fiber technology, allowing these mechanisms to be discarded after coming into contact with whole blood sample (605). By providing a disposable separation component such as cassette (617), the cleaning and sterilizing required when using blood-based diagnostic equipment is reduced. Cassette (617) may provide for multiple uses before being discarded.

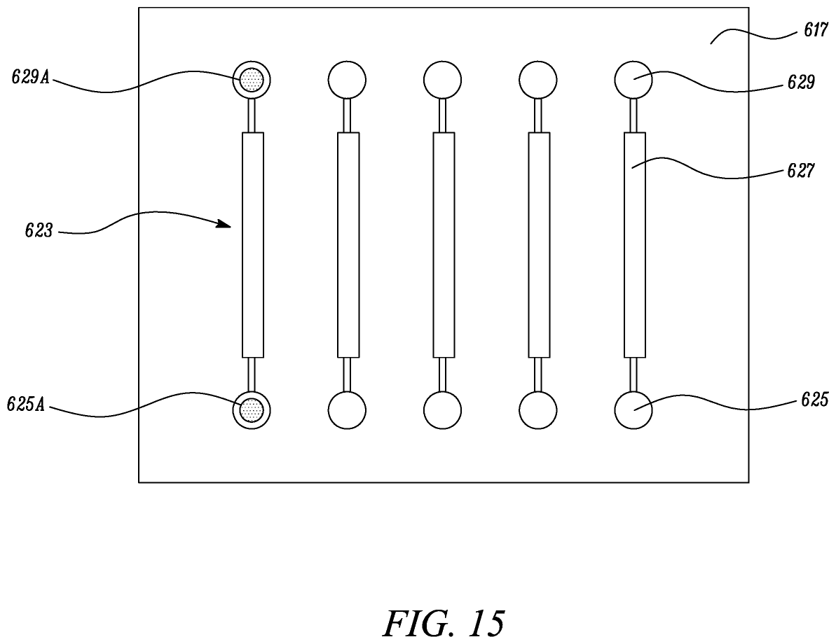

An exemplary cassette (617) is shown in greater detail in FIG. 15. Cassette (617) is configured to be removably received within cassette receptacle (615) of separation system (607). Some versions of cassette (617) are configured to receive all or a portion of whole blood sample (605) therein and create a separated sample, being either a plasma sample or a serum sample, from whole blood sample (605) without centrifugation. Some versions of cassette (617) include one or more separation channels (623). Each separation channel (623) extends from a first portion (625) to a second portion (627) to a third portion (629). In general, the portion of whole blood sample (605) is deposited in first portion (625) and travels into second portion (627) where a separated sample is generated therefrom. The separated sample thereafter travels into third portion (629) for further use by analyzer (601). While multiple separation channels (623) are shown in FIG. 15, cassette (617) may alternatively include one separation channel (623) per cassette (617). Alternatively, each separation channel (623) may produce multiple separated samples from whole blood sample (605). In some versions of separation channel (623), this is accomplished by having more than one third portion (629) per separation channel (623).

First portion (625) and third portion (629) may comprise wells such as those wells found in assay or other microbial instruments. First portion (625) and third portion (629) may include a film or other covering over unused wells in cassette (617). Third portion (629) may be an open or otherwise uncovered well. Separation system (607) is configured to puncture the film or covering of first portion (625) in order to open up the well for receiving the whole blood sample (605). Similarly, separation system (607) is configured to puncture the film or covering of third portion (629) in order to open up the well for removing the separated sample formed via the particular separation channel (623). As shown in FIG. 15, first portion (625A) and third portion (629A) are depicted as punctured and thus the associated separation channel (623) has been used by analyzer (601). Analyzer (601) will move to the next unused separation channel (623) when creation of a new separation sample is desired from either whole blood sample (605) or a different batch of whole blood. In this way, cassette (617) may be used to facilitate non-centrifugal separation of whole blood numerous times before being needing replaced and further eliminates the need for cleaning or sterilizing of separation equipment.

Second portion (627) generates a separated sample from the portion of whole blood sample (605) inserted into first portion (625) without centrifugation. Second portion (627) may include a filtration system such as a hollow fiber system and/or hollow fiber technology and may be configured to separate whole blood sample (605) into a plasma sample and/or a serum sample.

Movement system (609) is configured to transfer at least a portion of whole blood sample (605) from tube (603) to separation system (607). Movement system (609) includes a first transfer element (619) movable between tube (603) and separation system (607). First transfer element (619) is configured to obtain all or a portion of whole blood sample (605) from tube (603) and deposit it into separation system (607). First transfer element (619) may include a pipette or any other element or mechanism for drawing and depositing all or a portion of whole blood sample (605).

In some versions of analyzer (601), movement system (609) is configured to transfer at least a portion of the separated sample generated by separation system (607) from separation system (607) to analysis system (611). In some versions of analyzer (601), movement system (609) includes a second transfer element (621) movable between separation system (607) and analysis system (611). Second transfer element (621) is configured to obtain at least a portion of the separated sample from separation system (607) and deposit it into analysis system (611).

In other versions of analyzer (601), tube (603) may be placed directly onto an adapter (not shown) which draws the portion of whole blood sample (605) into separation system (607), without the need for first transfer element (619). Similarly, in some versions of analyzer (601), third portion (629) may provide the separated sample directly to analysis system (611) through features such as a cuvette or similar element, without the need for second transfer element (621).

VI. Exemplary Plasma Separation

Generating plasma without the use of centrifugation in conjunction and connection with analyzer (601) or any other biochemical analyzer which may require plasma samples generated from whole blood before the actual diagnostic analysis has numerous advantages. Currently, centrifugation creates a bottleneck in the laboratory workflow. Blood samples that need to be spun to generate plasma can take up to five to fifteen minutes onboard the centrifugate in order to generate a usable plasma. Further, once the centrifugation starts the laboratory technician is forced to wait until this batch is complete. This workflow causes delays and potentially leads to a delay of the results.

The common laboratory workflow requires the laboratory technician to perform various steps and can potentially be erroneous before loading the spun plasma sample onboard the instrument. From entry into the lab, this workflow involves: (a) accessioning samples; (b) sorting samples; (c) labeling samples; (d) racking samples; (e) centrifuge samples; (f) de-capping samples; (g) carrying samples to required workstations; (h) placing samples onboard instrument; (i) removing samples from instrument; (j) recapping samples; and (k) storing samples.

Centrifugation is currently the main method for generating plasma from blood. As detailed above, centrifugation takes at least five to fifteen minutes to generate plasma and requires several manual steps from the user. Centrifugation also requires batching the samples. Further, the centrifugation process may lead to sample hemolysis.

A. Plasma Separation Using Hollow Fiber Filtration

In some versions of separation system (607), plasma may be generated from whole blood using a filter system such as a hollow fiber filtration system. Hollow fiber filtration technology may be implemented into a consumable (i.e. disposable) element which is used in a specific device that can be directly or indirectly connected to analyzer (601) or another biochemical analyzer. In some versions, cassette (617) includes the hollow fiber filtration system and/or technology. In other versions, cassette (617) is replaced by using individual consumable elements such as a single use consumable using hollow fiber filtration system and/or technology or a grouping of hollow fiber filtration consumable elements which can produce plasma from multiple whole blood samples within the same consumable unit.

As shown in FIGS. 6A-6C, separation system (607) may comprise a plasma filtration system incorporating hollow fiber element (115). In general, hollow fiber element (115) receives as input whole blood and filters out plasma from the whole blood. In some versions of hollow fiber element (115), the walls of hollow fiber element (115) allow plasma to pass therethrough but retain or restrict the remainder of the whole blood from passing therethrough. Thus, hollow fiber element (115) separates the plasma from the whole blood for use as needed by analyzer (601). The plasma is thereafter collected from exterior surface (127) and the general exterior of hollow fiber element (115), effectively separating the plasma from the rest of the whole blood. This allows analyzer (601) to quickly produce plasma for further use therein without centrifugation. Details of hollow fiber element (115) has been discussed above with reference to FIGS. 6A-6C.

B. Stand-Alone Element

Some versions of analyzer (601) may be coupled with a stand-alone element (631) which may be separate from analyzer (601) or connected via a connection (633). Stand-alone element (631) encompasses an automated processing device which is able to process a consumable (635) of a specific size and generate plasma from whole blood while minimizing the manual interaction by the operator. Some versions of consumable (635) may incorporate hollow fiber filtration system and/or technology, such as those depicted in FIGS. 6A, 6B, and 6C.

Stand-alone element (631) may process capped sample tubes by removing or penetrating the cap of the sample tube and drawing a defined volume of whole blood from the tube. Stand-alone element (631) may be fitted with a de-capping mechanism. This mechanism is able to automatically remove caps from the sample tubes and discard the removed caps into a waste chute. Alternatively, stand-alone element (631) may be fitted with a cap piercing probe. The probe may be configured to directly pierce the cap of a blood tube and directly draw the blood, without the need to de-cap. Either syringe pump technology or pressure/vacuum driving pump technology may be used to draw the whole blood into the probe. The probe may be motor driven and capable of moving along all three axes (XYZ) and may undergo a wash cycle with an appropriate reagent after each sampling to minimize the likelihood of sample contamination. All of the aforementioned movement or transfer steps may be provided through movement system (609) or any other system within analyzer (601) as discussed above.

Once drawn, stand-alone element (631) dispenses the whole blood into sample reservoir of consumable (635). Thereafter, plasma is generated via consumable (635). In some versions of consumable (635), plasma is generated using hollow fiber filtration system and/or technology and associated features, such as one or more features depicted in hollow fiber element (115).

Once plasma is generated via consumable (635), the plasma is presented in one or several outlet reservoirs. In some versions of consumable (635), plasma is presented in several reservoirs to be able to generate aliquoting from the same sample if this is required by the operator or the underlying plasma testing. In some versions of stand-alone element (631), the outlet reservoir(s) of consumable (635) may be moved to an exposed position where it can be accessed by analyzer (601) for further use therein. Within stand-alone element (631), the whole blood samples and consumable (635) may be moved around by a sample transport module capable such as movement system (609) of accessing and moving both the sample tube and consumable (635). This can be accomplished by using a gantry system capable of moving along all three axes (XYZ). The gantry system can either make use of stepper motors in combination with a lead screw or a belt system, or it can also be powered by using a linear actuator. The sample transport module can also make use of a solenoid or an electromagnetic component to engage the sample tube and/or consumable (635). All of the aforementioned movement or transfer steps may be provided through movement system (609) or any other system within analyzer (601) as discussed above.

In those versions of consumable (635) utilizing hollow fiber filtration system and/or technology and associated features, once consumable (635) has been moved into a dedicated processing or buffer area, it will undergo plasma separation and this process will be automated by hollow fiber filtration system and/or technology. Consumables (635) and the hollow fiber elements can be mounted in a horizontal or a vertical orientation to accommodate for space and throughput constraints of the separation system (607).

C. Integrated Handler Element

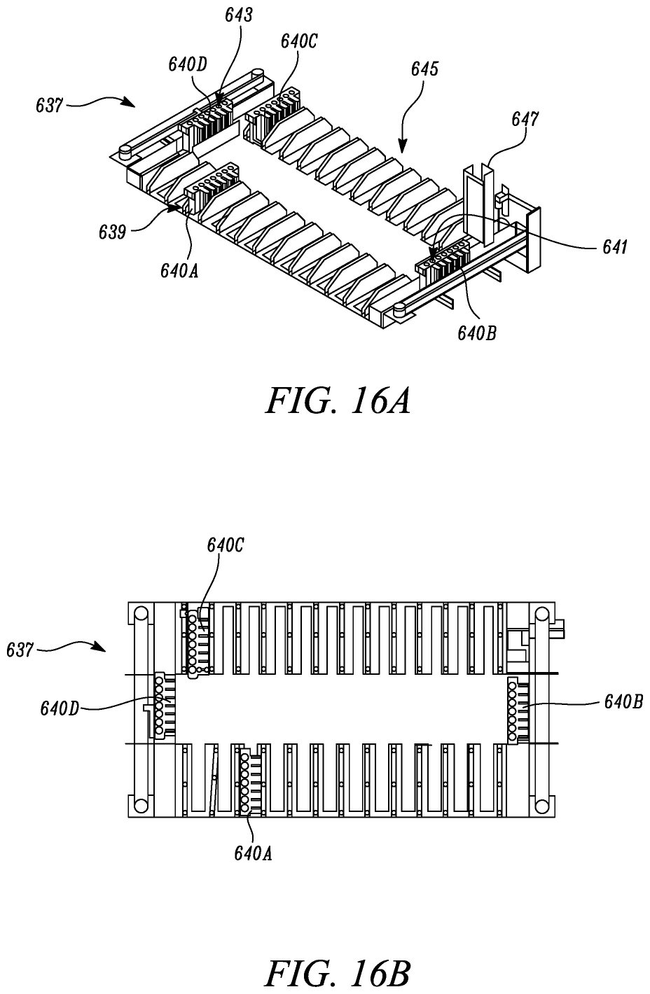

As shown in FIGS. 14 and 16A-16B, in those versions of analyzer (601) with a handler element for receiving racks of samples, an integrated handler element (637) with plasma separation features may be provided with features used to facilitate plasma generation. For example, handler element (637) may utilize hollow fiber filtration system and/or technology and thus include a hollow fiber element such as hollow fiber element (115), as shown in FIGS. 6A-6C.

Various features used in hollow fiber filtration system and/or technology may be placed above or in space between the rack tracking system of analyzer (601) or other analyzers and the offloading area. For example, separation system (607) may be placed above or in the space between the current rack tracking system and the offloading area. Corresponding software may be coupled with analyzer (601) to create a separate plasma sample type that includes specific parameters to allow differentiation of serum and plasma within the existing rack system.

As shown in FIGS. 16A-16B, integrated handler element (637) may include a rack loading station for plasma samples only, shown in FIG. 16A as a rack loader (639), with a dedicated barcode reader (not shown) associated with the plasma loading station. Integrated handler element (637) may include a first sampling area (641), a second sampling area (643), a rack buffer area (645), and a decapper (647). In some versions of integrated handler element (637), workflow proceeds such that a technician loads a rack (640) of whole blood sample tubes into rack loader (639), shown as rack (640A). Thereafter, the rack is moved to sampling area (641), shown as rack (640B), whereby the decapper (647) removes the cap of the sample tubes. Thereafter a portion of the whole blood sample for a particular tube may be placed into a consumable or otherwise processed to initialize the plasma generation process. In some versions of integrated handler element (637), the portion of whole blood is placed into a consumable associated with another rack and this rack is moved into the rack buffer area, shown as rack (640C). For those consumables using a hollow fiber filtration system and/or technology, while in the rack buffer area hollow fibers may be employed to generate plasma. Thereafter, once plasma is generated from a particular sample, the rack and/or sample is moved to sampling area (643), shown as rack (640D), for acquiring the plasma sample. In some versions of integrated handler element (637), upon starting analyzer (601), the separation system (607) would immediately start processing the plasma from the whole blood without waiting for the rest of the sub-systems of analyzer (601) to initialize. All of the aforementioned movement or transfer steps may be provided through movement system (609) or any other system within analyzer (601) as discussed above.

Integrated handler element (637) may include a large volume whole blood sample probe capable of picking up 300 μL or more. The probe may utilize disposable pipette tips that can be pre-loaded into analyzer (601). In some versions of analyzer (601), these disposable pipette tips are disposed under the rack loader elements. In some other versions of integrated handler element (637), no disposable tips are used and separation system (607) includes a wash station for washing and sterilizing the probe. In still other versions of integrated handler element (637), the standard sample probe may be used to withdraw plasma directly from the hollow fiber elements such as a hollow fiber collection cup. With respect to previous handler elements, integrated handler element (637) may include altered dimensions to increase collection capacity to 100 μL or more and may also have side cup feature added to collect plasma vertically.

Integrated handler element (637) may include updates or configuration regarding the placement, movement, and disposal of consumables such as a hollow fiber filtration system and/or technology. For example, horizontal or vertical pre-loaded blocks of individual or sheets of hollow fiber elements may be stacked and pushed into position with respect to the sample probes of the separation system (607) for whole blood dispensing. Thereafter, these hollow fiber elements may be utilized to produce plasma and thereafter moved into position for the main sample probe of analyzer (601). A queuing mechanism may be provided for those consumables and/or hollow fiber elements with plasma awaiting sampling to prevent backlog within analyzer (601).

Separation system (607) may include various timing considerations for maximizing efficient throughput from separating plasma, delivering plasma, and cleaning the plasma probe. For example, as consumables move from initial fill with whole blood to plasma sampling by the sample probe, this movement may be timed to allow for reagent and sample dispensing according to each underlying assay's settings. The placement of the consumable into final position under the sample probe can be the trigger for reagent dispensing for cleaning of the probe.

D. Integrated Wheel Element

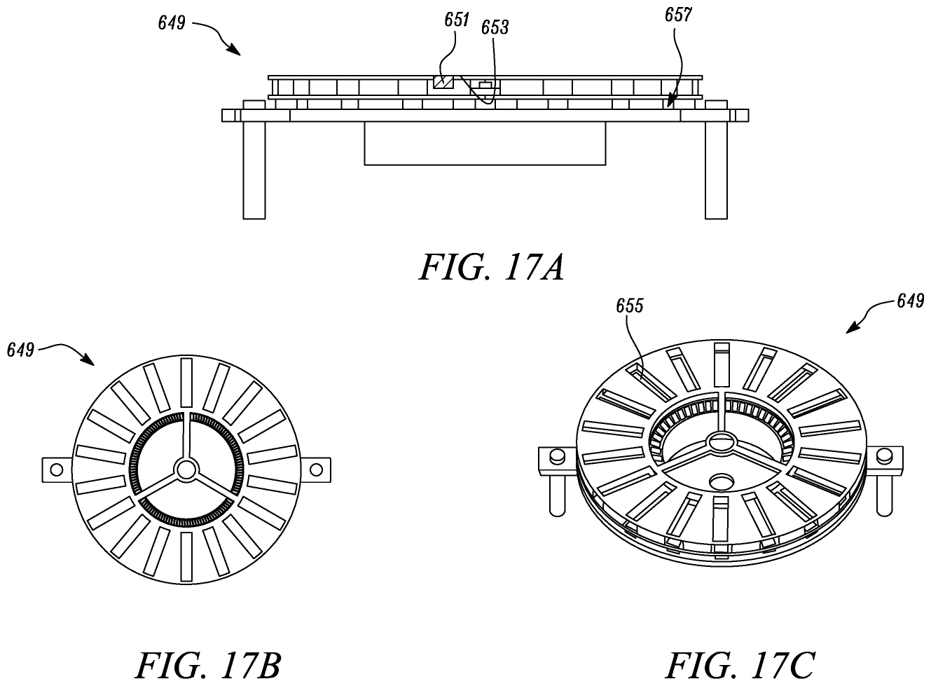

As shown in FIGS. 17A-17C, a hollow fiber filtration system and/or technology may be an integrated component of a reaction wheel (649) of analyzer (601). In some versions of analyzer (601), a reusable hollow fiber consumable (651) is provided within a holder (653) of reaction wheel (649) and may generate enough plasma to run the test directly in a cuvette (655) of reaction wheel (649). Reusable hollow fiber consumable (651) may be a reusable consumable that undergoes the same wash cycle as cuvette (655).

In these versions of analyzer (601), a tube containing a whole blood sample is loaded manually by the lab technician into a common rack. The common rack would be carried over to analyzer (601) and loaded into the sample loader space. The barcode in each sample will be read by the onboard barcode reader. In some versions of analyzer (601), a decapper is available and thus the sample tube would first be decapped onboard. In other versions of analyzer (601) a cap piercing probe is available and thus the next step will be performed by that probe. If neither a decapper nor a cap piercing probe is available, a user removes the cap.

An amount of whole blood from the sample is then aspirated to generate enough plasma for the specified test. The whole blood is thereafter delivered to reusable hollow fiber consumable (651) onboard reaction wheel (649) by a sample probe. In some versions of analyzer (601), the sample probe is capable of aspirating a minimum of 120 μL.

The plasma probe would subsequently be cleaned accordingly with the appropriate chemicals to minimize the chance of cross contamination of the next sample. Once the lifecycle of the reusable hollow fiber consumable (651) is complete, hollow fiber consumable (651) is ejected from a slot in reaction wheel (649) into a trash chute, and holder (653) is loaded with another reusable hollow fiber consumable (651). Analyzer (601) may also include a pump to deliver the correct volume of plasma to reaction wheel (649).

Once every tube in the rack has been sampled, that rack will go in the buffer area of the instrument until the results are released. Once the results are released, the rack is moved to the front loader where the customer can take the samples and spin them for storage. Reusable hollow fiber consumables (651) may be loaded into analyzer (601) as part of a cassette, which may be in the form of cassette (617). A user would load the cassette into analyzer (601) and refill as needed. All of the aforementioned movement or transfer steps may be provided through movement system (609) or any other system within analyzer (601) as discussed above.

E. Integrated Cap Element

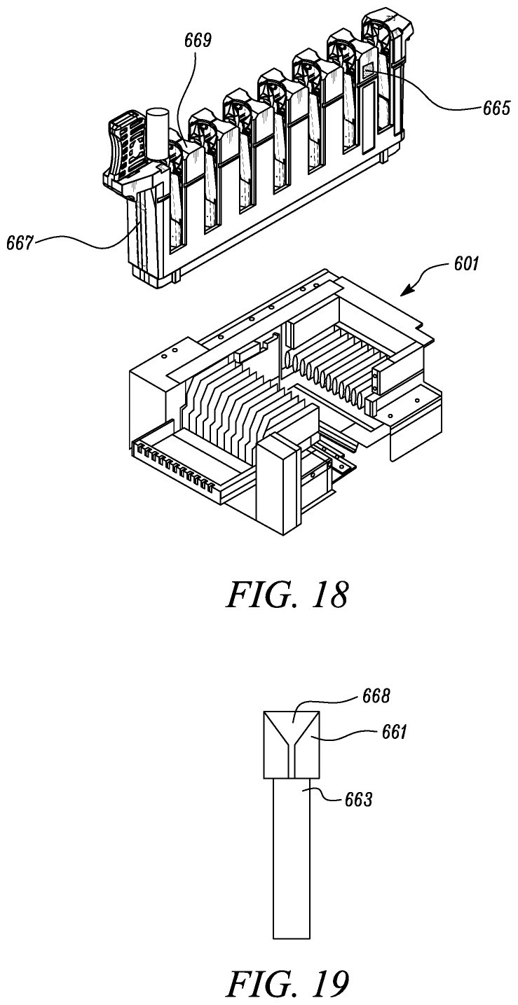

As shown in FIGS. 18 and 19, some versions of analyzer (601) may include features configured to retrieve plasma from the blood collection tubes placed therein. In these versions of analyzer (601), an insert (661) is connected to a blood collection tube (663). Once the whole blood is collected in blood collection tube (663), blood collection tube (663) is capped with insert (661). Insert (661) processes a portion of the whole blood inside blood collection tube (663) and converts this portion of the whole blood into plasma using a hollow fiber system and/or hollow fiber technology. The plasma is thereafter accumulated in the top of insert (661) in a reservoir (668) where it is available for a probe of analyzer (601). This probe retrieves an amount of the plasma from reservoir (668) of insert (661) and used it within analyzer (601) as needed.

In operation, blood collection tube (663) with insert (661) thereon is loaded into rack (667) and carried to analyzer (601) by a user. Rack (667) is thereafter loaded onboard analyzer (601) and moved to a buffer area where the whole blood inside blood collection tube (663) with insert (661) thereon undergoes plasma separation through agitation by transducer (665). Once plasma generation is been completed and an amount of plasma is accumulated within reservoir (668), the analyzer (601) would move the appropriate rack to get sampled by the sampling probe and deliver the specified volume of plasma to the reaction cuvette of analyzer (601) and the sample probe would be washed accordingly. All of the aforementioned movement or transfer steps may be provided through movement system (9) or any other system within analyzer (601) as discussed above.

F. Cassette Element

In some versions of analyzer (601), cassette (617) may incorporate a hollow fiber system and/or hollow fiber technology therein. For example, first portion (625), second portion (627), and/or third portion (629) of each separation channel (623) may include a filtration system such as a hollow fiber system and may be configured to separate whole blood sample (605) into a plasma sample and/or a serum sample.

G. Hollow Fiber Plasma Separation Module

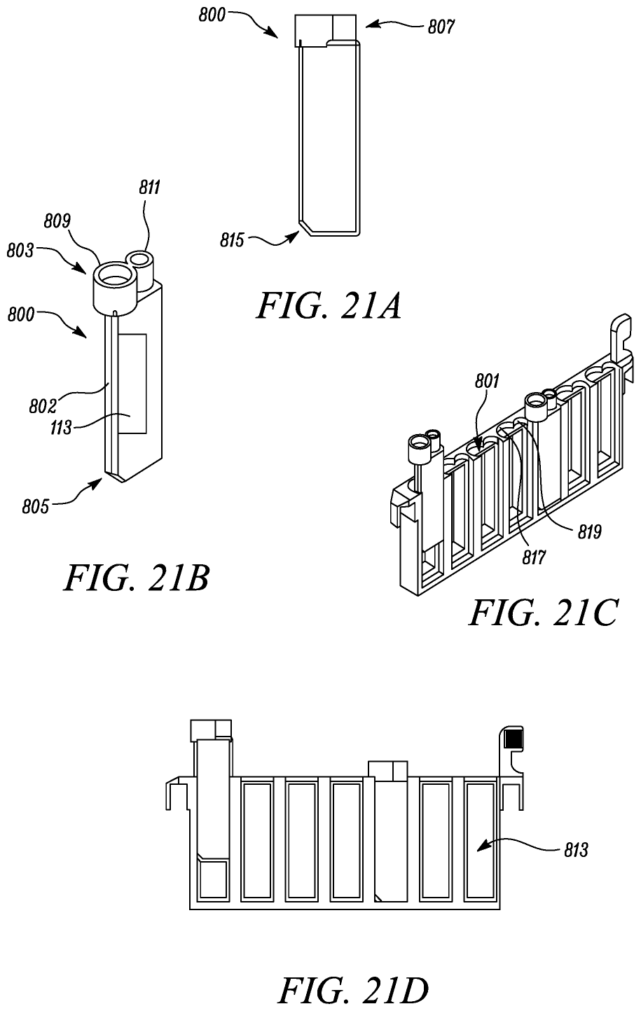

As shown in FIGS. 21A-21D, a hollow fiber plasma separation module (800) is provided along with a rack (801) and generally used to convert whole blood into plasma for further use within analyzer (601) by applying hollow fiber technology to whole blood deposited therein. In this configuration, portions of hollow fiber plasma separation module (800) generally comprise separation system (607). Hollow fiber plasma separation module (800) includes a hollow fiber system (113) with hollow fiber element (115) and/or filter wall (117) as described above incorporated therein to filter plasma from whole blood. In some versions of hollow fiber plasma separation module (800), one or more elements of hollow fiber system (113) is disposed within a housing (802) and used to convert whole blood into plasma within housing (802). For example, hollow fiber element (115) may be disposed within hollow fiber plasma separation module (800).

This configuration is similar to insert (661) as described above, wherein insert (661) receives whole blood inside insert (661) and passes the plasma generated therein to a collection tube connected with insert (661). Here, hollow fiber plasma separation module (800) receives the whole blood and passes it through housing (802) to generate plasma and present it or excrete it for further use within analyzer (601).

As shown in FIGS. 21A-21D, hollow fiber plasma separation module (800) extends from a top (803) to a bottom (805), with a well area (807) disposed at top (803). Well area (807) includes an input well (809) configured to receive whole blood therein. Input well (809) is operatively connected to hollow fiber system (113) disposed within housing (802) of hollow fiber plasma separation module (800), whereby the whole blood sample deposited in input well (809) is transferred through hollow fiber system (113) to generate plasma. While input well (809) is depicted as a cylindrical element in FIGS. 21A-21D, any orientation or shape of input well (809) may be used to facilitate the receipt of whole blood therein.

Well area (807) further includes an output well (811) configured to collect the plasma generated by hollow fiber system (113) within housing (802) and expose the plasma for further use as needed within analyzer (601). The plasma exposed within output well (811) may be collected by a plasma sampling probe or similar sampling element and delivered to a reaction cuvette of analyzer (601) or any other desired element within analyzer (601). While output well (811) is depicted as a cylindrical element in FIGS. 21A-21D, any orientation or shape of output well (811) may be used to facilitate the collection and exposure of plasma generated within housing (802).

In some versions of hollow fiber plasma separation module (800), housing (802) is generally a flat, rectangular shaped body to enable housing (802) to fit vertically within a corresponding and complementary shaped slot (813) of rack (801). A notch (815) is provided and defined by housing (802) to ensure proper insertion into slot (813) and orient input well (809) and output well (811) accordingly. A corresponding alignment feature is disposed (not shown) in the internal area defined by slot (813) to prevent hollow fiber plasma separation module (800) from being pressed fully into slot (813) when oriented incorrectly. Further, rack (801) defines an input well receiving space (817) and an output well receiving space (819). Both input well receiving space (817) and output well receiving space (819) are sized accordingly to receive their respective portions of well area (807) and ensure hollow fiber plasma separation module (800) is properly inserted into rack (801) in the correct orientation.

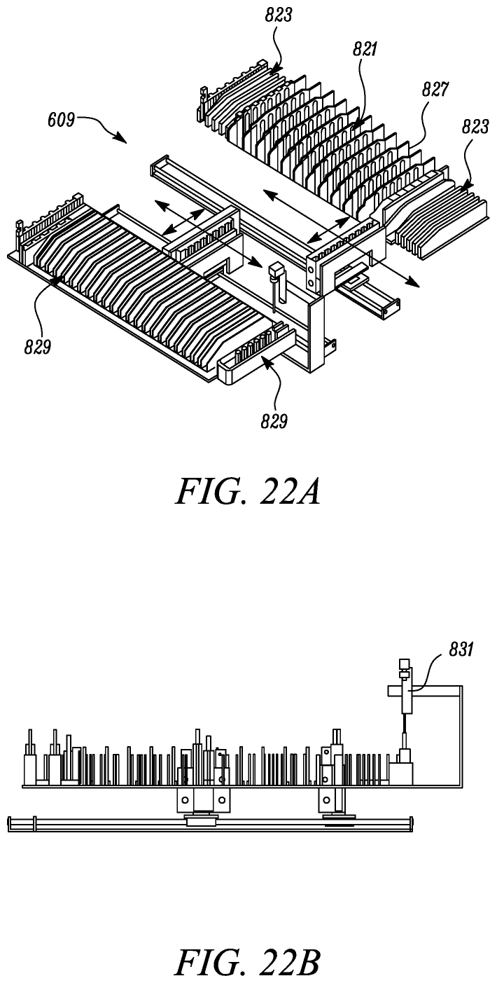

In the version of hollow fiber plasma separation module (800) depicted in FIGS. 22A-22B, a plasma separation module (821) includes a plurality of slots (823). Slots (823) are sized and configured to receive one rack (801) therein. Per configuration of analyzer (601), as needed, any additional mechanics or steps can be applied to each hollow fiber plasma separation module (800) in rack (801) to facilitate separating the plasma from the whole blood. Once plasma is produced, the rack (801) is moved from slot (823) to stand alone area (827), which is a series of slots sized to hold a rack (801) on plasma separation module (821) and await further movement within analyzer (601) as needed. Standalone area (827) may be used to hold empty racks (801) or racks (801) filled with yet to be used hollow fiber plasma separation modules (800). In some versions of analyzer (601) and separation system (607), a user may load hollow fiber plasma separation modules (800) directly onto a portion of plasma separation module (821) for use in analyzer (801).

A gantry or movement element such as that described above with respect to movement system (609) may be used to collect whole blood from a particular tube in a sample loading area (829) and deposit the whole blood in a particular hollow fiber plasma separation module (800) within a particular rack (801). As shown in FIGS. 22A-22B, a sample probe (831) may be used to transfer whole blood to input wells (809) of hollow fiber plasma separation modules (800).

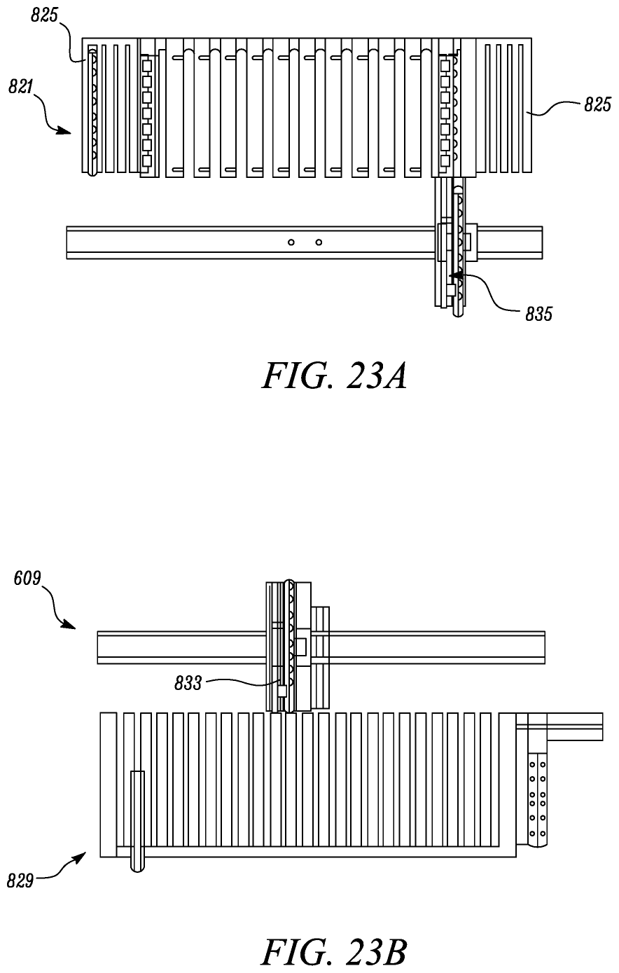

As shown in FIGS. 23A-23B, movement system (609) with respect to plasma separation module (821) may include two transport modules for moving blood sample racks and racks (801) with respect to the above steps and procedures. For example, movement system (609) may include a dual rack transport module (833) for receiving a blood sample rack thereon and a rack (801) with at least one unused hollow fiber plasma separation module (800) therein. Sample probe (831) (FIGS. 22A-22B) draws a portion of the whole blood from the tube associated with the blood sample rack in dual rack transport module (833) and deposits it into input well (809) of an unused hollow fiber plasma separation module (800). Thereafter, rack (801) is moved into a rack transport module (835), which places the associated rack (801) into a particular slot (823). Once the plasma is generated, rack transport module (835) moves rack (801) into standalone area (827) to await further use by analyzer (601).

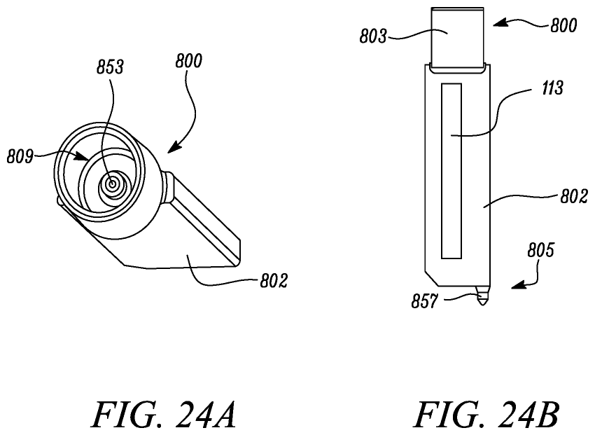

As shown in FIGS. 24A-24B, input well (809) may be formed with a needle (853) disposed therein. Needle (853) is utilized to puncture or otherwise penetrate a cap of a blood tube (not shown) to facilitate the transfer of whole blood into hollow fiber plasma separation module (800).

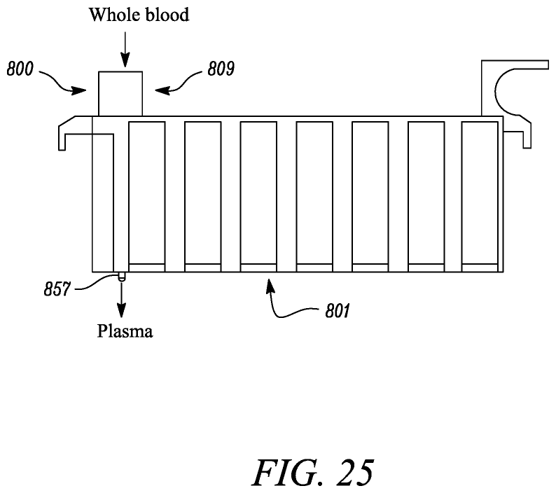

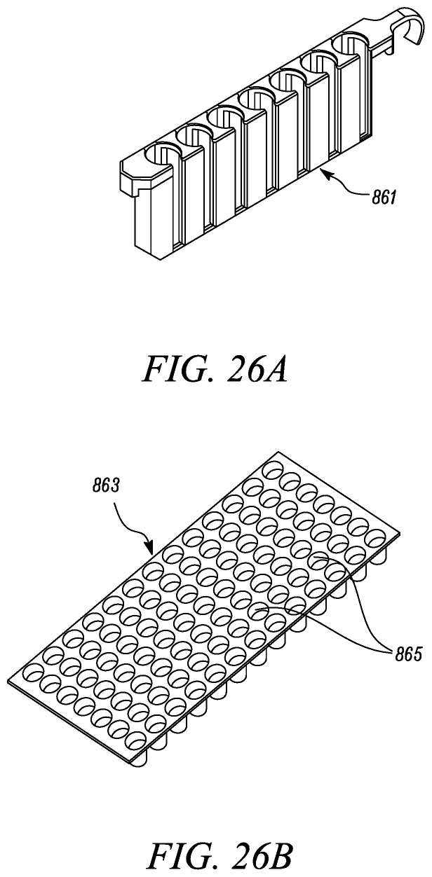

Rather than output well (811), as shown in FIGS. 24A-24B and 25, hollow fiber plasma separation module (800) may include a dispense tip (857) located at bottom (805) of housing (802). In this configuration, the whole blood input is disposed on one end of housing (802), while the plasma output is disposed on the opposite end of housing (802). Plasma created inside housing (802) by hollow fiber system (113) is dispensed out dispense tip (857), whereby it is collected by another element of analyzer (601) and used as needed. Accordingly, rack (801) may define an area (not shown) for receiving dispense tip (857) of each hollow fiber plasma separation module (800) disposed therein. Upon generation, plasma exits hollow fiber plasma separation modules (800) via dispenser tips (857) and drips into collection elements disposed below. Thereafter, the collected plasma is transferred as needed throughout analyzer (601) in furtherance of the analyzer workflow. As shown in FIGS. 26A-26B, a plasma sample rack (861) having a corresponding number of tubes therein (not shown) or a cuvette (863) having a corresponding number of deposit cells (865) may be disposed under each dispense tip (857) in a particular rack (801) to receive the plasma as it is produced.

VII. Exemplary Method of Analyzing a Whole Blood Sample

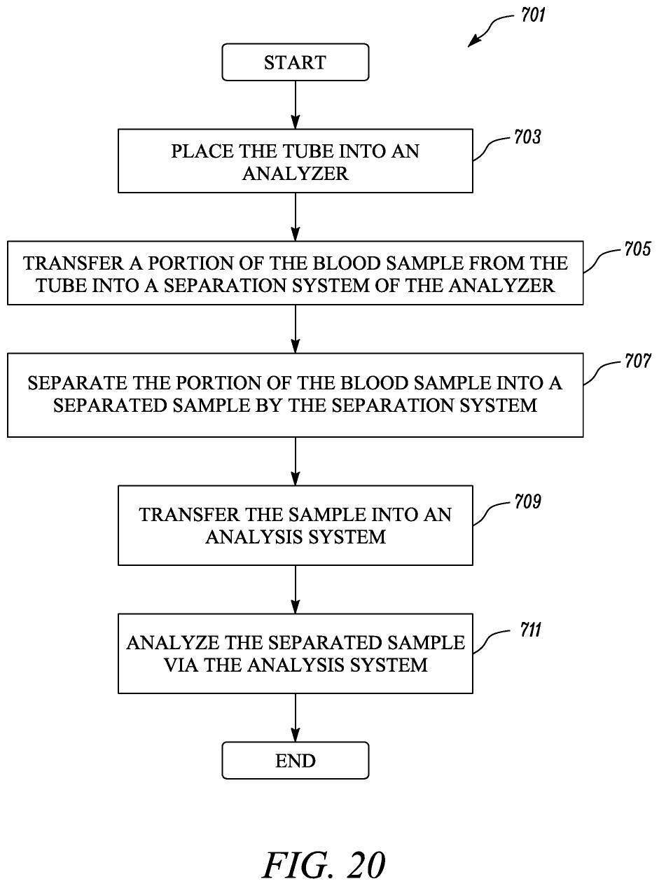

One method of analyzing a whole blood sample (701) using some of the elements described above is depicted generally in FIG. 20. Method of analyzing a blood sample (701) begins with a step (703), whereby a tube similar to tube (603) of FIG. 14 is placed into an analyzer similar to analyzer (601) of FIG. 14. The tube contains a whole blood sample from an individual. The placement of the tube may be performed by a nurse or phlebotomist or any other caregiver or clinician. Once the tube is placed into the analyzer, method of analyzing a blood sample (701) proceeds from step (703) to a step (705).

In step (705), all or some of the whole blood sample from the tube is transferred into a separation system of the analyzer, wherein the separation system is similar to separation system (607) of FIG. 14. The transfer of the whole blood sample from the tube to the separation system may be accomplished by a movement system similar to movement system (609) of FIG. 14. For example, a first transfer element such as a movable pipette may be used to draw all or some of the whole blood sample from the tube and deposit it into the separation system. After the whole blood sample is transferred from the tube to the separation system, method of analyzing a blood sample (701) proceeds from step (705) to a step (707).

In step (707), the whole blood sample deposited in the separation system is separated into a separated sample without the use of centrifugation. The separated sample may comprise plasma or serum. As described with separation system (607), the mechanism for creating the plasma sample may be in the form of a filtration system such as those incorporating a hollow fiber element or a similar system for creating a plasma sample from the whole blood sample. Hollow fiber element may incorporate a filter wall or other material for filtering plasma out of whole blood, thus producing plasma from whole blood without centrifugation.

Step (707) may make use of a cassette, similar to cassette (617) of FIGS. 14 and 15, to separate the whole blood sample into the separated sample. The cassette may be removable and replaceable within separation system and include one or more separation channels similar to separation channel (623) described above and depicted in FIG. 15. After the separated sample is separated from the whole blood sample, method of analyzing a blood sample (701) proceeds from step (707) to a step (709).

In step (709), the separated sample generated in step (707) is transferred into an analysis system similar to analysis system (611) as described above. The transfer of the separated sample from the separation system to the analysis system may be accomplished by a movement system similar to movement system (609) of FIG. 14. For example, a second transfer element such as a movable pipette may be used to draw the separated sample from the separation system and deposit it into the analysis system. After the separated sample is deposited in the analysis system, method of analyzing a blood sample (701) proceeds from step (709) to a step (711).

In step (711), the separated sample is analyzed by the analysis system. The analysis system may include a chemistry analyzer, an immunoassay analyzer, a molecular analyzer, a mass spectrometry analyzer, or a combination thereof. After the separated sample is analyzed by the analysis system, method of analyzing a blood sample (701) proceeds to end.

VIII. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A collection system comprising: (a) a tube; and (b) a holder, wherein the holder is configured to receive the tube therein.

### EXAMPLE 2

The collection system of Example 1 or any collection system or method of the previous subsequent Examples, wherein the tube comprises a plasma separation system.

### EXAMPLE 3

The collection system or method of any of the previous or subsequent Examples, wherein the plasma separation system comprises a filtration system.

### EXAMPLE 4

The collection system or method of any of the previous or subsequent Examples, wherein the filtration system comprises a hollow fiber element.

### EXAMPLE 5

The collection system or method of any of the previous or subsequent Examples, wherein the hollow fiber element comprises: (a) a filter wall, wherein the filter wall is configured to allow plasma to pass therethrough; and (b) an interior defined by the filter wall, wherein the interior is configured to received whole blood therein.

### EXAMPLE 6

The collection system or method of any of the previous or subsequent Examples, further comprising a pressure system configured to force whole blood through the filter wall.

### EXAMPLE 7

The collection system or method of any of the previous or subsequent Examples, wherein the tube defines a collection chamber.

### EXAMPLE 8

The collection system or method of any of the previous or subsequent Examples, wherein the collection chamber defines an analysis pocket therein.

### EXAMPLE 9

The collection system or method of any of the previous or subsequent Examples, wherein the holder comprises an imaging system.

### EXAMPLE 10

The collection system or method of any of the previous or subsequent Examples, wherein an optics element of the imaging system is directed at the analysis pocket when the tube is disposed in the holder.

### EXAMPLE 11

The collection system or method of any of the previous or subsequent Examples, wherein a portion of the tube adjacent the analysis pocket is transparent.

### EXAMPLE 12

The collection system or method of any of the previous or subsequent Examples, wherein the holder comprises a reagent reservoir, wherein the holder is configured to transfer a reagent from the reagent reservoir to the collection chamber.

### EXAMPLE 13

The collection system or method of any of the previous or subsequent Examples, wherein the tube comprises a label area on an outer surface.

### EXAMPLE 14

The collection system or method of any of the previous or subsequent Examples, wherein the holder comprises a labeling system, wherein the labeling system is configured to apply a label to the label area when the tube is disposed in the holder.

### EXAMPLE 15

The collection system or method of any of the previous or subsequent Examples, wherein the label area comprises a flat rectangular-shaped surface.

### EXAMPLE 16

The collection system or method of any of the previous or subsequent Examples, wherein the tube comprises a tube alignment element, wherein the holder includes a holder alignment element, wherein the tube alignment element is configured to cooperate with the holder alignment element when the tube is disposed in the holder.

### EXAMPLE 17

A collection system comprising: (a) a tube comprising: (i) a collection chamber, (ii) a plasma separation system, wherein the plasma separation system is disposed in the collection chamber, and (iii) an analysis pocket extending from the collection chamber, wherein the analysis pocket is configured to receive therein a unit of plasma provided from the plasma separation system; and (b) a holder configured to draw power from a power supply, the holder comprising: (i) a display screen, wherein the display screen in powered by the power supply; (ii) a tube receptacle, wherein the tube receptacle is configured to receive the tube therein, (iii) an analysis system, wherein the analysis system is configured to collect a set of data from the unit of plasma disposed in the analysis pocket, wherein the analysis system is configured to actuate the display screen to display the set of data, and (iv) a labeling system, wherein the labeling system is configured to apply a label to the tube, and (v) a pressure system configured to force fluid through a hollow fiber.

### EXAMPLE 18

The collection system or method of any of the previous or subsequent Examples, wherein the plasma separation system comprises a filtration system.

### EXAMPLE 19

The collection system or method of any of the previous or subsequent Examples, wherein the filtration system comprises a hollow fiber element.

### EXAMPLE 20

The collection system or method of any of the previous or subsequent Examples, the holder comprising a reagent reservoir, wherein the holder is configured to transfer a reagent from the reagent reservoir to the collection chamber.

### EXAMPLE 21

A method comprising: (a) placing a blood sample into a tube; (b) creating a plasma sample from the blood sample within the tube, wherein the plasma sample is created without centrifugation; (c) receiving the tube in a holder; and (d) conducting spectroscopic analysis on the sample of plasma with an analysis system of the holder.

### EXAMPLE 22

The collection system or method of any of the previous or subsequent Examples, further comprising creating the plasma sample via a plasma separation system disposed in the tube.

### EXAMPLE 23

The collection system or method of any of the previous or subsequent Examples, wherein the plasma separation system comprises a filtration system.

### EXAMPLE 24

The collection system or method of any of the previous or subsequent Examples, wherein the filtration system comprises a hollow fiber element.

### EXAMPLE 25

The collection system or method of any of the previous or subsequent Examples, further comprising forcing the blood sample through the hollow fiber element to create the plasma sample.

### EXAMPLE 26

The collection system or method of any of the previous or subsequent Examples, further comprising printing a label on the tube while the tube is disposed within the holder.

### EXAMPLE 27

The collection system or method of any of the previous or subsequent Examples, further comprising determining a sample quality of the sample based at least in part on the spectroscopic analysis.

### EXAMPLE 28

The collection system or method of any of the previous or subsequent Examples, further comprising indicating the sample quality to a user.

### EXAMPLE 29

The collection system or methods of any of the previous or subsequent Examples, further comprising indicating the sample quality to the user on a display screen of the holder.

### EXAMPLE 30

The collection system or methods of any of the previous or subsequent Examples, further comprising indicating the sample quality to the user on an indication system of the holder.

### EXAMPLE 31

The collection system or methods of any of the previous or subsequent Examples, further comprising providing the sample quality to a central analysis platform.

### EXAMPLE 32

The collection system or methods of any of the previous or subsequent Examples, further comprising labeling the tube while the tube is disposed within the holder.

### EXAMPLE 33

The collection system or methods of any of the previous or subsequent Examples, further comprising programming an RFID chip embedded within the tube while the tube is disposed within the holder.

### EXAMPLE 34

The collection system or methods of any of the previous or subsequent Examples, further comprising creating the plasma sample via a plasma separation system disposed in the tube.

### EXAMPLE 35

The collection system or methods of any of the previous or subsequent Examples, wherein the tube comprises an electronic label.

### EXAMPLE 36

The collection system or methods of any of the previous Examples, wherein the holder comprises a labeling system, wherein the labeling system is configured to program the electronic label when the tube is disposed in the holder.

### EXAMPLE 37

A device comprising: (a) a main body; (b) a needle attachment feature, wherein the needle attachment feature is configured to couple a needle element to the main body, wherein the needle attachment feature is configured to transmit a blood sample from the needle element into the main body; (c) a first chamber; (d) a second chamber; and (e) a separation element, wherein the separation element is configured to separate the blood sample into a first portion and a second portion, wherein the separation element is configured to transmit the first portion into the first chamber, wherein the separation element is configured to transmit the second portion into the second chamber.

### EXAMPLE 38

The device, system, or methods of the previous or subsequent Examples, wherein the separation element is disposed in the main body.

### EXAMPLE 39

The device, system, or methods of any of the previous or subsequent Examples, wherein one or both of the first chamber and the second chamber are removably secured to the main body.

### EXAMPLE 40

The device, system, or methods of any of the previous or subsequent Examples, wherein one or both of the first chamber and the second chamber comprise a test tube.

### EXAMPLE 41

The device, system, or methods of any of the previous or subsequent Examples, wherein one of the first chamber and the second chamber includes an interior surface, wherein the interior surface is coated with silica.

### EXAMPLE 42

The device, system, or methods of any of the previous or subsequent Examples, wherein one of the first chamber and the second chamber includes an interior surface, wherein the interior surface is coated with an anticoagulant.

### EXAMPLE 43

The device, system, or methods of any of the previous or subsequent Examples, wherein the separation element comprises a plasma separation system.

### EXAMPLE 44

The device, system, or methods of any of the previous or subsequent Examples, wherein the plasma separation system comprises a filtration system.

### EXAMPLE 45

The device, system, or methods of any of the previous or subsequent Examples, wherein the filtration system comprises a hollow fiber element.

### EXAMPLE 46

The device, system, or methods of any of the previous or subsequent Examples, wherein the hollow fiber element comprises: (a) a filter wall, wherein the filter wall is configured to allow plasma to pass therethrough; and (b) an interior defined by the filter wall, wherein the interior is configured to received whole blood therein.

### EXAMPLE 47

The device, system, or methods of any of the previous or subsequent Examples, wherein the separation element comprises one or more of a micro-channel, a filter, an anticoagulant, and a resin.

### EXAMPLE 48

The device, system, or methods of any of the previous or subsequent Examples, wherein the needle element comprises a blood draw needle.

### EXAMPLE 49

The device, system, or methods of any of the previous or subsequent Examples, wherein the needle element comprises a finger stick attachment.

### EXAMPLE 50

The device, system, or methods of any of the previous or subsequent Examples, comprising an identification element associated with the main body.

### EXAMPLE 51

The device, system, or methods of any of the previous or subsequent Examples, wherein the separation element is configured to separate the blood sample into the first portion based at least in part on the identification element.

### EXAMPLE 52

The device, system, or methods of any of the previous or subsequent Examples, wherein the first chamber is removably secured to the main body.

### EXAMPLE 53

A method comprising: (a) drawing a blood sample into a device; (b) separating the blood sample into a sample of whole blood, a sample of plasma, and a sample of serum; (c) transmitting the sample of whole blood into a first chamber connected to the device; (d) transmitting the sample of plasma into a second chamber connected to the device; and (e) transmitting the sample of serum into a third chamber connected to the device.

### EXAMPLE 54

The device, system, or methods of any of the previous or subsequent Examples, further comprising disconnecting one or more of the first chamber, the second chamber, and the third chamber from the device.

### EXAMPLE 55

The device, system, or methods of any of the previous or subsequent Examples, further comprising: (a) aspirating the blood sample through a needle element into a separation element of the device; and (b) transmitting the sample of blood through the separation element to separate the blood sample into the sample of whole blood, the sample of plasma, and the sample of serum.

### EXAMPLE 56

The device, system, or methods of any of the previous or subsequent Examples, wherein the needle element comprises a blood draw needle.

### EXAMPLE 57

The device, system, or methods of any of the previous or subsequent Examples, wherein the needle element comprises a finger stick attachment.

### EXAMPLE 58

The device, system, or methods of any of the previous or subsequent Examples, further comprising coating an interior surface of the first chamber with an anticoagulant.

### EXAMPLE 59

The device, system, or methods of any of the previous or subsequent Examples, further comprising coating an interior surface of the third chamber with silica.

### EXAMPLE 60

The device, system, or methods of any of the previous or subsequent Examples, further comprising: (a) prior to drawing the blood sample into the device, connecting a syringe to the device; (b) in response to connecting the syringe to the device, creating a vacuum in the device.

### EXAMPLE 61

The device, system, or methods of any of the previous or subsequent Examples, further comprising after transmitting the sample of whole blood into a first chamber connected to the device, the sample of plasma into a second chamber connected to the device, and the sample of serum into a third chamber connected to the device, disconnecting the syringe from the device.

### EXAMPLE 62

The device, system, or methods of any of the previous or subsequent Examples, further comprising after disconnecting the syringe from the device, loading the device into a sample rack.

### EXAMPLE 63

The device, system, or methods of any of the previous or subsequent Examples, further comprising inserting a first portion of the device into a slot defined by the sample rack to load the device into the sample rack, wherein the syringe is connected to a first portion of the device.

### EXAMPLE 64

A system comprising: (a) a device comprising: (i) a main body, (ii) a needle attachment feature, wherein the needle attachment feature is configured to selectively couple a needle element to the main body, wherein the needle attachment feature is configured to transmit a blood sample from the needle element into the main body, and (iii) a separation element disposed inside the main body, wherein the separation element is configured to receive the blood sample and separate the blood sample into a sample of whole blood, a sample of plasma, and a sample of serum; (b) a first sample tube, wherein the first sample tube is removably connected to the device, wherein the first sample tube is configured to receive the sample of whole blood from the device; (c) a second sample tube, wherein the second sample tube is removably connected to the device, wherein the second sample tube is configured to receive the sample of plasma from the device; and (d) a third sample tube, wherein the third sample tube is removably connected to the device, wherein the third sample tube is configured to receive the sample of serum from the device.

### EXAMPLE 65

The device, system, or methods of any of the previous or subsequent Examples, wherein an interior surface of the first sample tube includes a layer of anticoagulant.

### EXAMPLE 66

The device, system, or methods of any of the previous or subsequent Examples, wherein an interior surface of the third sample includes a layer of silica.

### EXAMPLE 67

The device, system, or methods of any of the previous or subsequent Examples, wherein the separation element comprises one or more of a micro-channel, a filter, an anticoagulant, and a resin.

### EXAMPLE 68

The device, system, or methods of any of the previous or subsequent Examples, wherein the separation element comprises a plasma separation system.

### EXAMPLE 69

The device, system, or methods of any of the previous or subsequent Examples, wherein the plasma separation system comprises a filtration system.

### EXAMPLE 70

The device, system, or methods of any of the previous or subsequent Examples, wherein the filtration system comprises a hollow fiber element.

### EXAMPLE 71

The device, system, or methods of any of the previous or subsequent Examples, wherein the hollow fiber element comprises: (a) a filter wall, wherein the filter wall is configured to allow plasma to pass therethrough; and (b) an interior defined by the filter wall, wherein the interior is configured to received whole blood therein.

### EXAMPLE 72

The device, system, or methods of any of the previous or subsequent Examples, wherein the needle element comprises a blood draw needle.

### EXAMPLE 73

The device, system, or methods of any of the previous Examples, wherein the needle element comprises a finger stick attachment.

### EXAMPLE 74

An analyzer configured to receive a tube containing a blood sample, the analyzer comprising: (a) a separation system; (b) a transfer element, wherein the transfer element is configured to obtain at least a portion of the blood sample from the tube, wherein the transfer element is configured to deposit the portion of the blood sample into the separation system; (c) a movement system, wherein the movement system is configured to move the transfer element between the tube and the separation system; and wherein the separation system is configured to separate the portion of the blood sample into a separated sample, wherein the sample comprises one of a plasma sample and a serum sample.

### EXAMPLE 75

The analyzer, analyzer system, cassette, and/or method of the previous or any of the subsequent Examples, wherein the transfer element is a first transfer element and further comprising: (a) an analysis system; (b) a second transfer element, wherein the second transfer element is configured to obtain at least a portion of the separated sample from the separation system, wherein the second transfer element is configured to deposit the portion of the separated sample into the analysis system; and wherein the movement system is configured to move the second transfer element between the separation system and the analysis system.

### EXAMPLE 76

The analyzer, analyzer system, cassette, and/or method of any of the previous or subsequent Examples, wherein the analysis system comprises one of a spectroscopic analyzer, a thermocycler element for PCR, an isotheral amplification element, an immune assay, and an Elisa system.

### EXAMPLE 77

The analyzer, analyzer system, cassette, and/or method of any of the previous or subsequent Examples, wherein one of the first transfer element and the second transfer element comprise a pipette.

### EXAMPLE 78

The analyzer, analyzer system, cassette, and/or method of any of the previous or subsequent Examples, wherein the separation system comprises a filtration system.

### EXAMPLE 79

The analyzer, analyzer system, cassette, and/or method of any of the previous or subsequent Examples, wherein the filtration system comprises a hollow fiber element.

### EXAMPLE 80

The analyzer, analyzer system, cassette, and/or method of any of the previous or subsequent Examples, wherein the hollow fiber element comprises: a filter wall, wherein the filter wall is configured to allow plasma to pass therethrough; and (b) an interior defined by the filter wall, wherein the interior is configured to received whole blood therein.

### EXAMPLE 81

The analyzer, analyzer system, cassette, and/or method of any of the previous or subsequent Examples, wherein the separation system defines a cassette receptacle, wherein the cassette receptacle is configured to receive a cassette therein.

### EXAMPLE 82

An analyzer system comprising: (a) a cassette; and (b) an analyzer configured to receive a tube containing a blood sample and the cassette, the analyzer comprising: (i) a separation system; (ii) a transfer element, wherein the transfer element is configured to obtain at least portion of the blood sample from the tube, wherein the transfer element is configured to deposit the portion of the blood sample into the separation system; (iii) a movement system, wherein the movement system is configured to move the transfer element between the tube and the separation system; (iv) a cassette receptacle defined by the separation system, wherein the cassette receptacle is configured to removably receive the cassette therein; and wherein the separation system is configured to separate the portion of the blood sample into a separated sample via the cassette, wherein the separated sample comprises one of a plasma sample and a serum sample.

### EXAMPLE 83

The analyzer, analyzer system, cassette, and/or method of any of the previous or subsequent Examples, wherein the cassette comprises a plurality of separation channels, wherein the separation system is configured to separate the portion of the blood sample into the separated sample via one of the plurality of separation channels.

### EXAMPLE 84

The analyzer, analyzer system, cassette, and/or method of any of the previous or subsequent Examples, wherein each separation channel in the plurality of separation channels comprises: (a) a first portion, wherein the first portion is configured to receive the portion of the blood sample therein; and (b) a second portion, wherein the second portion is configured to receive the portion of the blood sample from the first portion and separate the portion of the blood sample into the separated sample.

### EXAMPLE 85

The analyzer, analyzer system, cassette, and/or method of any of the previous or subsequent Examples, wherein the separation system comprises a filtration system.

### EXAMPLE 86

The analyzer, analyzer system, cassette, and/or method of any of the previous or subsequent Examples, wherein the filtration system comprises a hollow fiber element.

### EXAMPLE 87

The analyzer, analyzer system, cassette, and/or method of any of the previous or subsequent Examples, wherein the microfluidic separation system comprises a lateral cavity acoustic transducer system.

### EXAMPLE 88

The analyzer, analyzer system, cassette, and/or method of any of the previous or subsequent Examples, wherein the hollow fiber element comprises (a) a filter wall, wherein the filter wall is configured to allow plasma to pass therethrough; and (b) an interior defined by the filter wall, wherein the interior is configured to received whole blood therein.

### EXAMPLE 89

A method of analyzing a blood sample in a tube, the method comprising: (a) placing the tube into an analyzer; (b) transferring at least a portion of the blood sample from the tube into a separation system of the analyzer, wherein the transferring is performed automatically within the analyzer; (c) separating the portion of the blood sample into a separated sample by the separation system, wherein the separated sample is one of a plasma sample and a serum sample; (d) transferring the separated sample into an analysis system, wherein the transferring is performed automatically within the analyzer; and (e) analyzing the separated sample via the analysis system.

### EXAMPLE 90

The analyzer, analyzer system, cassette, and/or method of any of the previous or subsequent Examples, wherein the separation system comprises a removable cassette, and further comprising transferring the portion of the blood sample from the tube into the cassette.

### EXAMPLE 91

The analyzer, analyzer system, cassette, and/or method of any of the previous or subsequent Examples, wherein the cassette comprises at least one sealed well, and further comprising: (a) puncturing one of the at least one sealed wells to form a punctured well; and (b) transferring the portion of the blood sample from the tube into the punctured well.

### EXAMPLE 92

The analyzer, analyzer system, cassette, and/or method of any of the previous or subsequent Examples, wherein the cassette comprises a separation portion and at least one open well, and further comprising moving the portion of the blood sample from the punctured well into the separation portion and toward one of the at least one open wells to separate the portion of the blood sample into the separated sample.

### EXAMPLE 93

The analyzer, analyzer system, cassette, and/or method of any of the previous or subsequent Examples, wherein the separation system comprises a filtration system.

### EXAMPLE 94

The analyzer, analyzer system, cassette, and/or method of any of the previous or subsequent Examples, wherein the filtration system comprises a hollow fiber element.

### EXAMPLE 95

The analyzer, analyzer system, cassette, and/or method of any of the previous or subsequent Examples, wherein the hollow fiber element comprises: (a) a filter wall, wherein the filter wall is configured to allow plasma to pass therethrough; and (b) an interior defined by the filter wall, wherein the interior is configured to received whole blood therein.

### EXAMPLE 96

The analyzer, analyzer system, cassette, and/or method of any of the previous or subsequent Examples, further comprising: (a) transferring the portion of the blood sample from the tube into a first portion of the cassette; (b) separating the portion of the blood sample into the separated sample in a second portion of the cassette; and (c) transferring the separated sample from a third portion of the cassette into the analysis system.

### EXAMPLE 97

The analyzer, analyzer system, cassette, and/or method of any of the previous or subsequent Examples, further comprising puncturing the first portion prior to transferring the portion of the blood sample from the tube into the first portion of the cassette.

### EXAMPLE 98

The analyzer, analyzer system, cassette, and/or method of any of the previous or subsequent Examples, wherein the second portion comprises one of a microfluidic separation system, a hollow-fiber filtering system, and a lateral cavity acoustic transducer.

### EXAMPLE 99

The analyzer, analyzer system, cassette, and/or method of any of the previous or subsequent Examples, wherein the cassette comprises a plurality of separation channels, wherein each separation channel in the plurality of separation channels comprises a first portion and a second portion, and further comprising: (a) selecting a separation channel from the plurality of separation channels; (b) transferring the portion of the blood sample from the tube into the first portion of the selected separation channel; and (c) separating the portion of the blood sample into the separated sample in the second portion of the selected separation channel.

### EXAMPLE 100

The analyzer, analyzer system, cassette, and/or method of any of the previous or subsequent Examples, wherein each separation channel in the plurality of separation channels comprises a third portion, and further comprising transferring the separated sample from the third portion of the selected separation channel into the analysis system.

### EXAMPLE 101

The analyzer, analyzer system, cassette, and/or method of any of the previous or subsequent Examples, further comprising puncturing the first portion of the selected separation channel prior to transferring the portion of the blood sample from the tube into the first portion of the selected separation channel.

### EXAMPLE 102

The analyzer, analyzer system, cassette, and/or method of any of the previous or subsequent Examples, wherein the second portion comprises one of a microfluidic separation system, a hollow-fiber filtering system, and a lateral cavity acoustic transducer.

### EXAMPLE 103

The analyzer, analyzer system, cassette, and/or method of any of the previous or subsequent Examples, further comprising manually removing the cassette from the analyzer.

### EXAMPLE 104

A cassette configured to be selectively disposed in an analyzer, the cassette comprising:

(a) a main body; (b) at least one separation channel disposed within the main body, wherein each separation channel is configured to separate at least a portion of a blood sample into a separated sample.

### EXAMPLE 105

The analyzer, analyzer system, cassette, and/or method of any of the previous or subsequent Examples, wherein each separation channel comprises: (a) a first portion, wherein the first portion is configured to receive the portion of the blood sample therein; and (b) a second portion, wherein the second portion is configured to receive the portion of the blood sample from the first portion and separate the portion of the blood sample into the separated sample.

### EXAMPLE 106

The analyzer, analyzer system, cassette, and/or method of any of the previous or subsequent Examples, wherein the second portion comprises a filtration system.

### EXAMPLE 107

The analyzer, analyzer system, cassette, and/or method of any of the previous or subsequent Examples, wherein the filtration system comprises a hollow fiber element.

### EXAMPLE 108

The analyzer, analyzer system, cassette, and/or method of any of the previous or subsequent Examples, wherein the hollow fiber element comprises: (a) a filter wall, wherein the filter wall is configured to allow plasma to pass therethrough; and (b) an interior defined by the filter wall, wherein the interior is configured to received whole blood therein.

### EXAMPLE 109

The analyzer, analyzer system, cassette, and/or method of any of the previous or subsequent Examples, wherein the first portion is a sealed well configured to be punctured prior to receiving the portion of the blood sample therein.

### EXAMPLE 110

The analyzer, analyzer system, cassette, and/or method of any of the previous or subsequent Examples, wherein each separation channel comprises a third portion configured to receive the separated sample from the second portion.

### EXAMPLE 111

The analyzer, analyzer system, cassette, and/or method of any of the previous or subsequent Examples, wherein the third portion is an open well.

### EXAMPLE 112

The analyzer, analyzer system, cassette, and/or method of any of the previous or subsequent Examples, wherein the separated sample is at least 10 microliters.

### EXAMPLE 113

The analyzer, analyzer system, cassette, and/or method of any of the previous or subsequent Examples, further comprising separating the portion of the blood sample into multiple separated samples by the separation system.

### EXAMPLE 114

The analyzer, analyzer system, cassette, and/or method of any of the previous or subsequent Examples, further comprising transferring a first separated sample in the multiple separated samples into a chemistry analyzer, wherein the transferring of the first separated sample is performed automatically within the chemistry analyzer.

### EXAMPLE 115

The analyzer, analyzer system, cassette, and/or method of any of the previous or subsequent Examples, further comprising transferring a second separated sample in the multiple separated samples into an immunoassay analyzer, wherein the transferring of the second separated sample is performed automatically within the immunoassay analyzer.

### EXAMPLE 116

The analyzer, analyzer system, cassette, and/or method of any of the previous or subsequent Examples, further comprising transferring a third separated sample in the multiple separated samples into a molecular analyzer, wherein the transferring of the third separated sample is performed automatically within the molecular analyzer.

### EXAMPLE 117

The analyzer, analyzer system, cassette, and/or method of any of the previous or subsequent Examples, further comprising transferring a fourth separated sample in the multiple separated samples into a mass spectrometry analyzer, wherein the transferring of the fourth separated sample is performed automatically within the spectrometry analyzer.

### EXAMPLE 118

The analyzer, analyzer system, cassette, and/or method of any of the previous or subsequent Examples, further comprising transferring a second portion of the blood sample from the tube into a storage container.

### EXAMPLE 119

The analyzer, analyzer system, cassette, and/or method of any of the previous or subsequent Examples, further comprising transferring at least portion of the separated sample into a storage container.

### EXAMPLE 120

An analyzer configured to receive a tube containing a blood sample, the analyzer comprising: (a) a separation system, wherein the separation system comprises a filtration system, wherein the filtration system comprises a hollow fiber element; (b) a transfer element, wherein the transfer element is configured to obtain at least a portion of the blood sample from the tube, wherein the transfer element is configured to deposit the portion of the blood sample into the separation system; (c) a movement system, wherein the movement system is configured to move the transfer element between the tube and the separation system; and wherein the separation system is configured to separate the portion of the blood sample into a separated sample, wherein the sample comprises one of a plasma sample and a serum sample.

### EXAMPLE 121

The analyzer, analyzer system, cassette, and/or method of any of the previous or subsequent Examples, wherein the separation system comprises a hollow fiber plasma separation module.

### EXAMPLE 122

The analyzer, analyzer system, cassette, and/or method of any of the previous or subsequent Examples, wherein the hollow fiber plasma separation module comprises: (a) a housing; (b) an input well defined by the housing; and (c) a hollow fiber element disposed in the housing and configured to generate plasma from whole blood received in the input well.

### EXAMPLE 123

The analyzer, analyzer system, cassette, and/or method of any of the previous or subsequent Examples, wherein the hollow fiber plasma separation module comprises an output well defined by the housing and configured to collect plasma generated by the hollow fiber element.

### EXAMPLE 124

The analyzer, analyzer system, cassette, and/or method of any of the previous or subsequent Examples, wherein the housing extends from a top end to a bottom end, wherein the input well is disposed at the top end, wherein the output well is disposed at the top end.

### EXAMPLE 125

The analyzer, analyzer system, cassette, and/or method of any of the previous or subsequent Examples, wherein the hollow fiber plasma separation module comprises a dispense tip extending from the housing and configured to dispense plasma generated by the hollow fiber element.

### EXAMPLE 126

The analyzer, analyzer system, cassette, and/or method of any of the previous or subsequent Examples, wherein the housing extends from a top end to a bottom end, wherein the input well is disposed at the top end, wherein the dispense tip is disposed at the bottom end.

### EXAMPLE 127

A method of analyzing a blood sample in a tube, the method comprising: (a) placing the tube into an analyzer; (b) transferring at least a portion of the blood sample from the tube into a separation system of the analyzer, wherein the transferring is performed automatically within the analyzer, wherein the separation system comprises a filtration system, wherein the filtration system comprises a hollow fiber element; (c) separating the portion of the blood sample into a separated sample by the separation system, wherein the separated sample is one of a plasma sample and a serum sample; (d) transferring the separated sample into an analysis system, wherein the transferring is performed automatically within the analyzer; and (e) analyzing the separated sample via the analysis system.

### EXAMPLE 128

The analyzer, analyzer system, cassette, and/or method of any of the previous or subsequent Examples, wherein the separation system comprises a hollow fiber plasma separation module.

### EXAMPLE 129

The analyzer, analyzer system, cassette, and/or method of any of the previous or subsequent Examples, further comprising: (a) receiving whole blood in an input well defined by a housing of the hollow fiber plasma separation module; (b) transferring the whole blood from the input well to a hollow fiber element disposed in the housing; and (c) generating plasma from the whole blood via the hollow fiber element.

### EXAMPLE 130

The analyzer, analyzer system, cassette, and/or method of any of the previous or subsequent Examples, further comprising collecting the plasma generated by the hollow fiber element in an output well defined by the housing.

### EXAMPLE 131

The analyzer, analyzer system, cassette, and/or method of any of the previous or subsequent Examples, wherein the housing extends from a top end to a bottom end, wherein the input well is disposed at the top end, wherein the output well is disposed at the top end.

### EXAMPLE 132

The analyzer, analyzer system, cassette, and/or method of any of the previous or subsequent Examples, further comprising dispensing plasma generated via the hollow fiber element through a dispense tip extending from the housing.

### EXAMPLE 133

The analyzer, analyzer system, cassette, and/or method of any of the previous or subsequent Examples, wherein the housing extends from a top end to a bottom end, wherein the input well is disposed at the top end, wherein the dispense tip is disposed at the bottom end.

IX. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A collection system comprising:
(a) a tube; and
(b) a holder, wherein the holder is configured to receive the tube therein.

2. The collection system of claim 1, wherein the tube comprises a plasma separation system.

3. The collection system of claim 2, wherein the plasma separation system comprises a filtration system.

4. The collection system of claim 3, wherein the filtration system comprises a hollow fiber element.

5. The collection system of claim 4, wherein the hollow fiber element comprises:
(a) a filter wall, wherein the filter wall is configured to allow plasma to pass therethrough; and
(b) an interior defined by the filter wall, wherein the interior is configured to received whole blood therein.

6. The collection system of claim 5, further comprising a pressure system configured to force whole blood through the filter wall.

7. The collection system of claim 1, wherein the tube defines a collection chamber.

8. The collection system of claim 7, wherein the collection chamber defines an analysis pocket therein.

9. The collection system of claim 1, wherein the holder comprises an imaging system.

10. The collection system of claim 9, wherein an optics element of the imaging system is directed at the analysis pocket when the tube is disposed in the holder.

11. The collection system of claim 8, wherein a portion of the tube adjacent the analysis pocket is transparent.

12. The collection system of claim 7, wherein the holder comprises a reagent reservoir, wherein the holder is configured to transfer a reagent from the reagent reservoir to the collection chamber.

13. The collection system of claim 1, wherein the tube comprises a label area on an outer surface.

14. The collection system of claim 13, wherein the holder comprises a labeling system, wherein the labeling system is configured to apply a label to the label area when the tube is disposed in the holder.

15. The collection system of claim 13, wherein the label area comprises a flat rectangular-shaped surface.

16. The collection system of claim 1, wherein the tube comprises a tube alignment element, wherein the holder includes a holder alignment element, wherein the tube alignment element is configured to cooperate with the holder alignment element when the tube is disposed in the holder.

17. A collection system comprising:
(a) a tube comprising:
(i) a collection chamber,
(ii) a plasma separation system, wherein the plasma separation system is disposed in the collection chamber, and
(iii) an analysis pocket extending from the collection chamber, wherein the analysis pocket is configured to receive therein a unit of plasma provided from the plasma separation system; and
(b) a holder configured to draw power from a power supply, the holder comprising:
(i) a display screen, wherein the display screen is powered by the power supply;
(ii) a tube receptacle, wherein the tube receptacle is configured to receive the tube therein,
(iii) an analysis system, wherein the analysis system is configured to collect a set of data from the unit of plasma disposed in the analysis pocket, wherein the analysis system is configured to actuate the display screen to display the set of data,
(iv) a labeling system, wherein the labeling system is configured to apply a label to the tube, and,
(v) a pressure system configured to force fluid through a hollow fiber.

18. The collection system of claim 17, wherein the plasma separation system comprises a filtration system.

19. The collection system of claim 18, wherein the filtration system comprises a hollow fiber element.

20. (canceled)

21. A method comprising:
(a) placing a blood sample into a tube;
(b) creating a plasma sample from the blood sample within the tube, wherein the plasma sample is created without centrifugation;
(c) receiving the tube in a holder; and
(d) conducting spectroscopic analysis on the sample of plasma with an analysis system of the holder.

22.-131. (canceled)
